(19) United States
(12) Patent Application Publication
(10) Pub. No.: US 2014/0178344 A1
(43) Pub. Date: Jun. 26, 2014

(54) ACRYLAMIDE HYDROGELS FOR TISSUE ENGINEERING

(71) Applicants: William B. Carlson, Seattle, WA (US); Gregory D. Phelan, Corltnad, NY (US)

(72) Inventors: William B. Carlson, Seattle, WA (US); Gregory D. Phelan, Corltnad, NY (US)

(73) Assignee: Empire Technology Development LLc

(21) Appl. No.: 13/879,976

(22) PCT Filed: Dec. 4, 2012
(86) PCT No.: PCT/US12/67782

§ 371 Date: Apr. 17, 2013

(51) Int. Cl. A61L 27/16; A61L 27/54; A61L 27/38; A61L 27/52; A61L 27/44

(52) U.S. Cl. CPC A61L 27/16; A61L 27/52; A61L 27/44; A61L 27/38; A61L 27/54
USPC 424/93.7; 523/113

(57) ABSTRACT

Disclosed are acrylamide hydrogels prepared from derivatives of (tetrahydropyranyl)methyl- and (tetrahydrofuranyl)methyl-acrylamide, methods of making the same, and methods of wound healing or tissue generation with acrylamide hydrogels.

### FIELD

The present technology is generally related acrylamide hydrogels, methods of making acrylamide hydrogels, and methods of wound healing or tissue regeneration with acrylamide hydrogels.

### BACKGROUND

Hydrogel polymers derived from acrylate monomers such as 2-hydroxyethylmethacrylate (HEMA) or acrylate-functionalized derivatives of poly(ethylene glycol) (e.g., poly(ethylene glycol) methacrylate, PEGMA) have been employed as scaffold materials for living cells. Likewise, polylactic acid (PLA) and poly(glycolic/glutaric acid-co-lactic acid) (PGLA) have been widely used as scaffold materials. Despite the widespread use of HEMA, PGLA, and other polymers in the biomedical field, there remain problems, particularly when such hydrogels are introduced in living systems. For example, polymeric materials derived from monomers such as HEMA or PEGMA may release toxic ethylene glycol into the body. Similarly, PLA and PGLA may release potent organic acids which may cause inflammatory responses and/or tumor growth. Furthermore, while hydrogels derived from PGLA, PLA and HEMA may initially display good flexibility properties, over time such hydrogels may degrade with concomitant loss in flexibility. Due to the tremendous potential of cell growth scaffolds to impact wound healing and health, there is considerable interest in the development of new scaffold materials.

### SUMMARY

The present technology provides acrylamide hydrogels which may be used as scaffold materials in numerous applications including, but not limited to, wound healing, tissue regeneration and engineering, artificial organ growth, and cartilage repair. In this regard, the acrylamide hydrogels can be used as scaffold materials in vivo to aid in the vascularization and creation of new tissues, the repair of wounded tissue, and to replace non-functional tissues. The hydrogels include may be prepared from inexpensive and renewable resources such as furanose and pyranose sugars and derivatives thereof using practical and scalable methods. The non-toxic biodegradation products from the acrylamide hydrogels-sugars and sugar-like derivatives—are fully compatible with, and may be metabolized by, living systems, thus eliminating in vivo toxicity and autoimmune response concerns associated with existing hydrogel scaffolds. Being comprised of sugar and sugar-like derivatives, the present hydrogels may also find use as cell culture media or use in other cell culture applications, since the hydrogels can provide an energy source to cells. For example, cancer cells may be entrained in the present hydrogels and cultured as part of research into the behavior of such cells in a three-dimensional environment. In addition to biocompatibility with cells and living tissues, the present hydrogels are characterized by mechanical robustness, and as such may be introduced in living systems where physical degradation of the scaffold is of concern.

In accordance with one aspect, the present technology provides a hydrogel including a polyacrylamide, where the polyacrylamide includes one or more repeating units derived from the compound of Formula I:

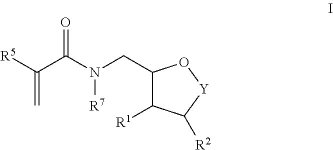

and stereoisomers thereof, where Y is —C(H)(R³)— or —C(H)(R³)—C(H)(R⁴)—; R¹, R². R³, and R⁴ are independently an —OH, a protected hydroxyl group, or a group of Formula II:

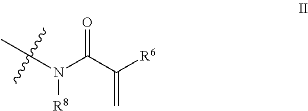

provided that not more than one of R¹, R², R³, and R⁴ is a group of Formula II; and R⁵, R⁶R⁷ and R⁸ are independently selected from hydrogen and a substituted or unsubstituted alkyl group. In some embodiments of the hydrogel derived from the compound of Formula I, R⁵ and R⁶ are independently selected from hydrogen or a methyl group. In certain embodiments R⁷ and R⁸ are independently hydrogen, a methyl group, or an ethyl group.

In some embodiments of the hydrogel derived from the compounds of Formula I, Y is —C(H)(R³)—C(H)(R⁴)—. In some embodiments, the protected hydroxyl group is selected from trimethylsilyl, t-butyldimethylsilyl, acetyl, benzyl, benzoyl, or methoxymethyl. In some embodiments, one, two, three, or four of R¹, R², R³, and R⁴ are an —OH.

The phrase “derived from” means that a hydrogel of the present technology is formed from, or otherwise prepared from, one or more compounds of Formula I, such as through a polymerization reaction. In this regard, one or more repeat units of the one or more compounds of Formula I may be incorporated into the present polymers. For example, and as will be appreciated by those of skill in the art, polymerization of one or more compounds of Formula I may provide a polymer which includes one or more of the following repeat units, where Y, R¹-R², R⁵, and R⁷ are defined as above:

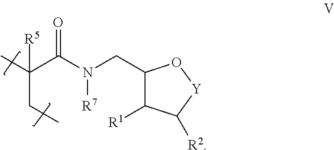

For clarity, it is understood that the above repeat unit may, but need not be, bonded to itself. For example, where one or more compounds of Formula I are copolymerized with one or more other monomers (not of Formula I), the above repeating unit may or may not be separated by a repeating unit of the one or more other monomers, i.e., random, block, or alternating copolymers may be formed. In the case of a homopolymer derived of one compound of Formula I, or a copolymer derived from more than one compound of Formula I, such a repeat unit may be bonded to itself. As will also be appreciated by those of skill in the art, where the one or more compounds of Formula I include two acrylamide groups, (i.e., where one of R¹, R², R³, and R⁴ is a group of Formula II), polymerization may proceed through either, or both, of the acrylamide groups.

In some embodiments, the one or more repeating units of the polyacrylamide are derived from the compound of Formula IA, IB or both IA and IB (where the variables are as defined above):

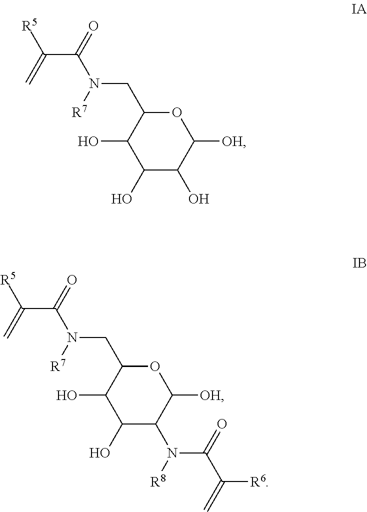

In some embodiments, the polyacrylamide includes about 90 wt % to about 99.9 wt % repeating units derived from the compound of Formula IA and about 0.1 wt % to about 10 wt % repeating units derived from the compound of Formula IB, based on the total weight of the compounds of Formula IA and IB. In some embodiments of the polyacrylamide derived from compounds of Formula I and IA, R⁵ is H and others R⁵ is methyl. In some embodiments of the polyacrylamide derived from compounds of Formula I and IA, R⁷ is H. In some embodiments of the polyacrylamide derived from compounds of Formula I and IB, R⁶ is H and in others R⁶ is methyl. In still other embodiments of polyacrylamide derived from compounds of Formula I and IB, one of R⁵ and R⁶ is H and one is methyl. In other embodiments of polyacrylamide derived from compounds of Formula I and IB, both R⁵ and R⁶ are H or both are methyl. In some embodiments of polyacrylamide derived from the compounds of Formula I and IB, R⁷ is H. In certain embodiments, R⁸ is H. In some embodiments, both R⁷ and R⁸ are H.

In certain embodiments, the one or more repeating units of the hydrogel are derived from one or more compounds selected from N-((3,4,5,6-tetrahydroxytetrahydro-2H-pyran-2-yl)methyl)acrylamide, N-((3,4,5,6-tetrahydroxytetrahydro-2H-pyran-2-yl)methyl)methacrylamide, N-((3,4,6-trihydroxy-5-methacrylamidotetrahydro-2H-pyran-2-yl)methyl)methacrylamide, N-((5-acrylamido-3,4,6-trihydroxytetrahydro-2H-pyran-2-yl)methyl)methacrylamide, N-(6-(acrylamidomethyl)-2,4,5-trihydroxytetrahydro-2H-pyran-3-yl)methacrylamide, or N-((5-acrylamido-3,4,6-trihydroxytetrahydro-2H-pyran-2-yl)methyl)acrylamide.

Any of the hydrogels of the present technology may further include collagen, drugs, and/or cells. In one embodiment, the hydrogel includes one or more drugs entrained within the hydrogel. In such an embodiment, the one or more drugs may be selected from anticancer agents, anti-inflammatory drugs, antibacterials, antivirals, or growth factors. In this regard, the present technology also provides a method of releasing a drug from the hydrogels described herein.

In another embodiment, the hydrogels of the present technology include one or more entrained cells. In some such embodiments, the cells are selected from any of those listed in Table I, below. In some embodiments the cells are selected from the group consisting of bone marrow, neuroblastoma, adenocarcinoma, carcinoma, colorectal carcinoma, glioblastoma, fibroblast, and myeloblast cells.

In another embodiment, the hydrogels of the present technology include interconnected cavities of about 0.1 μm to about 100 μm in dimension. In one such embodiment, the cavities are spherical pores having diameters of about 0.1 μm to about 100 μm.

In accordance with another aspect, the present technology provides a method of wound healing or tissue regeneration, the method including implanting any of the hydrogels described herein into a patient in need thereof. In one embodiment, the method includes implanting a hydrogel which further includes one or more drugs entrained within the hydrogel into a patient in need thereof. In another embodiment, the method includes implanting a hydrogel which further includes cells entrained within the hydrogel into a patient in need thereof.

According to another aspect, the present technology provides a method of making a hydrogel, the method including polymerizing one or more compounds of Formula I or stereoisomers thereof in an aqueous solution to form the hydrogel, where Formula I is

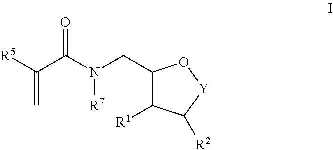

Y is —C(H)(R³)— or —C(H)(R³)—C(H)(R⁴)— and R¹, R², R³, and R⁴ are independently an —OH, a protected hydroxyl group, or a group of Formula II:

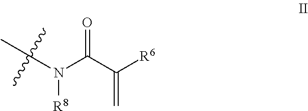

provided that not more than one of R¹, R², R³, and R⁴ is a group of Formula II; and R⁵, R⁶ R⁷ and R⁸ are independently selected from hydrogen and a substituted or unsubstituted alkyl group. In some embodiments, R⁵ and R⁶ are independently selected from hydrogen or a methyl group; and in some embodiments, R⁷ and R⁸ are independently selected from hydrogen, a methyl group or an ethyl group.

In one embodiment of the present methods, the one or more compounds of Formula I are selected from N-((3,4,5,6-tetrahydroxytetrahydro-2H-pyran-2-yl)methyl)acrylamide, N-((3,4,5,6-tetrahydroxytetrahydro-2H-pyran-2-yl)methyl)methacrylamide, N-((3,4,6-trihydroxy-5-methacrylamidotetrahydro-2H-pyran-2-yl)methyl)methacrylamide, N-((5-acrylamido-3,4,6-trihydroxytetrahydro-2H-pyran-2-yl)methyl)methacrylamide, N-(6-(acrylamidomethyl)-2,4,5-trihydroxytetrahydro-2H-pyran-3-yl)methacrylamide, or N-((5-acrylamido-3,4,6-trihydroxytetrahydro-2H-pyran-2-yl)methyl)acrylamide. In another embodiment, the one or more compounds of Formula I are selected from compounds of Formula IA, IB or both IA and IB:

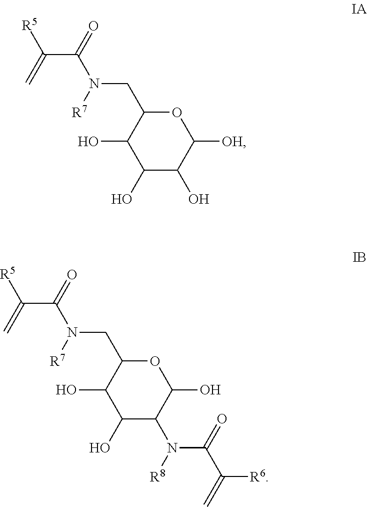

In one such embodiment, the hydrogel includes from about 90 wt % to about 99.9 wt % of the compound of Formula IA and from about 0.1 wt % to about 10 wt % of the compound of Formula IB, based on the total weight of the compounds of Formula IA and IB.

In another embodiment, the method includes carrying out the polymerization in the presence of collagen. In another embodiment, the polymerization is carried out in a vessel including acrylic microspheres having diameters of about 0.1 μm to 100 μm. In one such embodiment, the acrylic microspheres are leached from or removed from the hydrogel by exposing the hydrogel to an organic solvent in which the microspheres are soluble. In a further embodiment, the organic solvent is selected from one or more of acetone, dichloromethane, and ethanol. Finally, any of the polymerization methods described herein may be a high internal phase emulsion polymerization.

According to another aspect, the present technology provides a non-woven fibrous mat of electrospun polyacrylamide where the polyacrylamide includes one or more repeating units derived from the compound of Formula I:

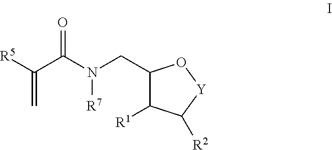

and stereoisomers thereof, where Y is —C(H)(R³)— or —C(H)(R³)—C(H)(R⁴)—; R¹, R², R³, and R⁴ are independently an —OH, a protected hydroxyl group, or a group of Formula II:

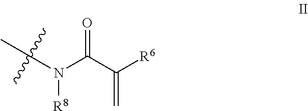

provided that not more than one of R¹, R², R³, and R⁴ is a group of Formula II; and R⁵, R⁶, R⁷ and R⁸ are independently selected from the group consisting of hydrogen and a substituted or unsubstituted alkyl group. In some embodiments, R⁵ and R⁶ are independently selected from hydrogen or a methyl group; and in certain embodiments, R⁷ and R⁸ are independently hydrogen, a methyl group or an ethyl group. In one embodiment, the non-woven fibrous mat of includes cells entrained within the mat. In one such embodiment, the cells are selected from the group consisting of bone marrow, neuroblastoma, adenocarcinoma, carcinoma, colorectal carcinoma, glioblastoma, fibroblast, and myeloblast cells.

According to yet another aspect, the present technology provides a method of wound healing or tissue regeneration, the method including implanting the non-woven fibrous mat into a patient in need thereof.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIG. 1 is a schematic illustration of the preparation of N-((3,4,5,6-tetrahydroxytetrahydro-2H-pyran-2-yl)methyl)acrylamide and N-((3,4,5,6-tetrahydroxytetrahydro-2H-pyran-2-yl)methyl)methacrylamide, according to an illustrative embodiment.

FIG. 2 is a schematic illustration of the preparation of N-((3,4,6-trihydroxy-5-methacrylamidotetrahydro-2H-pyran-2-yl)methyl)methacrylamide and N-((5-acrylamido-3,4,6-trihydroxytetrahydro-2H-pyran-2-yl)methyl)acrylamide, according to an illustrative embodiment.

FIG. 3 is a schematic illustration of the preparation of poly(N-((3,4,6-trihydroxy-5-methacrylamidotetrahydro-2H-pyran-2-yl)methyl)methacrylamide) and poly(N-((5-acrylamido-3,4,6-trihydroxytetrahydro-2H-pyran-2-yl)methyl)acrylamide), according to an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The technology is described herein using several definitions, as set forth throughout the specification.

The use of the terms “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In general, “substituted” refers to a group, as defined below (e.g., an alkyl or aryl group) in which one or more bonds to a hydrogen atom contained therein are replaced by a bond to non-hydrogen or non-carbon atoms or to carbon atoms bearing one or more heteroatoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, aroyloxyalkyl, heterocyclyloxy, and heterocyclylalkoxy groups; carbonyls (oxo), acyl; carboxyls; esters; urethanes; oximes; hydroxylamine; alkoxyamines; aralkoxyamines; thiols; sulfides; sulfoxides; sulfones; sulfonyls; sulfonamides; amines; N-oxides; hydrazines; hydrazides; hydrazones; azides; amides; thioamides; ureas; amidines; guanidines; enamines; imides; isocyanates; isothiocyanates; cyanates; thiocyanates; imines; nitro groups; nitriles; and the like. Such groups may be pendant or integral to the carbon chain itself. Cyclic groups (e.g., cycloalkyl, aryl, heterocyclyl) may also be substituted by carbon-based groups such as alkyl, alkenyl, and alkynyl, any of which may also be substituted (e.g., haloalkyl, hydroxyalkyl, aminoalkyl, haloalkenyl, and the like).

Alkyl groups include straight chain and branched alkyl groups having from 1 to 20 carbon atoms or, in some embodiments, from 1 to 12, 1 to 8, 1 to 6, or 1, 2, 3, or 4 carbon atoms. Alkyl groups further include cycloalkyl groups. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above. For example, the term haloalkyl refers to an alkyl group substituted with one or more halogen atoms.

Alkenyl groups include straight and branched chain and cycloalkyl groups as defined above, except that at least one double bond exists between two carbon atoms in the group. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, to 20 carbon atoms, 5 to 10 carbon atoms, or even 5, 6, 7, or 8 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 20 carbon atoms, 7 to 14 carbon atoms or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-ethyl-indanyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass unsaturated, partially saturated and saturated ring systems, such as, for example, imidazolyl, imidazolinyl and imidazolidinyl groups. The phrase “heterocyclyl group” includes fused ring species including those including fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyL benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyi groups. In some embodiments, heterocyclyl groups include saturated and unsaturated rings, but not aromatic rings. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, 4-ethyl-morpholinyl, 4-propylmorpholinyl, furan-2-yl methyl, furan-3-yl methyl, pyridine-3-yl methyl, tetrahydrofuran-2-yl ethyl, and indol-2-yl propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Alkoxy and aryloxy groups are hydroxyl groups (—OH) in which the bond to the hydrogen atom is replaced by a bond to a carbon atom of an alkyl group or aryl group, respectively, as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of aryloxy groups include but are not limited to phenoxy, naphthyloxy, and the like. Representative substituted alkoxy groups or aryloxy groups may be substituted one or more times with substituents such as those listed above.

Alkenoxy, alkynoxy, aralkyloxy, heterocyclyloxy, and heterocyclylalkoxy groups are defined analogously to alkoxy and aryloxy groups. Hence, alkenoxy, alkynoxy, aralkyloxy, heterocyclyloxy, and heterocyclylalkoxy groups are hydroxyl groups (—OH) in which the bond to the hydrogen atom is replaced by a bond to a carbon atom of, respectively, and alkene, alkyne, arylalkyl, heterocyclyl, or heterocyclylalkyl group as defined above. Representative substituted alkenoxy, alkynoxy, aralkyloxy, heterocyclyloxy, and heterocyclylalkoxy groups may be substituted one or more times with substituents such as those listed above.

The term “acrylamide group” or “acrylamido group” refers to RC(O)NR′— groups, where R is a substituted or unsubstituted alkenyl group as defined herein and R′ is H, or a substituted or unsubstituted alkyl, alkenyl, or aryl group. Representative acrylamide groups include, but are not limited to H₂C═CHC(O)NH—, H₂C═C(CH₃)C(O)NH—, H₂C═C(CH₂CH₃)C(O)NH—, H₃CCH═CHC(O)NH—, and the like. The term “acrylamide group” specifically includes methacrylamide groups (and methacrylamido groups) such as H₂C═C(CH₃)C(O)NH—.

The term “acrylate reagent” refers to a reagent with the formula RC(O)-LG, where R is a substituted or unsubstituted alkenyl group as defined herein and LG is a leaving group as defined herein. Non-limiting examples of acrylate reagents include acryloyl chloride and methacryloyl chloride.

The term “acryloyl” or “acryl” refers to RC(O)— groups, where R is a substituted or unsubstituted alkenyl group as defined herein. Representative acryloyl groups include, but are not limited to H₂C═CHC(O)—, H₂C═C(CH₃)C(O)—, H₂C═C(CH₂CH₃)C(O)—, H₃CCH═CHC(O)—, and the like.

The term “acyl” refers to RC(O)— groups, where R is a substituted or unsubstituted alkyl group as defined herein. Representative acyl groups include, but are not limited to, acetyl (CH₃C(O)—), and the like.

The term “amine” (or “amino”) as used herein refers to —NHR and —NRR′ groups, wherein R, and R′ are independently hydrogen, or a substituted or unsubstituted alkyl, alkenyl, alkynyl, aryl, or aralkyl group as defined herein. Non-limiting examples of amine groups include, but are not limited to, —NH₂, methylamino, dimethylamino, ethylamino, diethylamino, propylamino, isopropylamino, phenylamino, benzylamino, and the like.

The term “aroyl” refers to RC(O)— groups, where R is a substituted or unsubstituted aryl group as defined herein. Representative aroyl groups include, but are not limited to, benzoyl (PhC(O)—), and the like.

The term “base” refers to any chemical species, ionic or molecular, organic or inorganic, capable of accepting or receiving a proton (hydrogen ion) from another substance, generally an acid. The greater the tendency to accept a proton, the stronger the base. Representative bases include, but are not limited to: alkali or alkaline hydroxides (e.g., lithium hydroxide, sodium hydroxide, calcium hydroxide), hydrogencarbonates (e.g., sodium bicarbonate), carbonates (e.g., potassium carbonate), fluorides (e.g., potassium fluoride), alkoxides (e.g., sodium methoxide, potassium tert-butoxide), oxides (e.g. sodium oxide, magnesium oxide), hydrides (e.g., lithium hydride, calcium hydride), amides (e.g., sodium amide, lithium bis(trimethylsilylamide), lithium diisopropylamide), alkyls (e.g., butyllithium, tert-butyllithium); ammonia; alkylamines (e.g., trimethylamine, triethylamine, diisopropylethylamine); pyridines (e.g., 2,6-dimethylaminopyridine, pyridine), phosphazenes, amidines, guanidines, and the like.

The term “carboxyl” (or “carboxylic acid”) refers to —COOH.

The term “collagen” as used herein means any and all kinds of collagen, of any type, from any source, whether or not purified or made non-antigenic, without limitation. Cross-linked collagen, esterified collagen, and chemically-modified collagen are included with the term “collagen.”

The term “cyano” (or “nitrile”) refers to —CN groups.

The term “drug” as used herein refers to any substance which in vivo is capable of producing a desired, usually beneficial, effect and may be a drug having a therapeutic or prophylactic effect. The term explicitly includes a substance which itself does not produce a desired, usually beneficial, effect, but is metabolized or otherwise altered to form a drug, i.e., the term includes prodrugs. Non-limiting examples of drugs include, but are not limited to, antibiotics, antibacterials, anticancer agents, anti-inflammatories, analgesics, antivirals, antifungals, growth factors, vitamins, nutrients, and the like.

The term “ester” as used herein refers to —COOR groups, where R is a substituted or unsubstituted alkyl, alkenyl, alkynyl, or aryl group as defined herein.

The term “furanose” as used herein refers to carbohydrates and substituted derivatives of carbohydrates that include a tetrahydrofuran ring. Non-limiting examples of furanoses include, but are not limited to, arabinose, lyxose, ribose, xylose, and the like.

The term “halogen” (or “halo”) refers to —F, —Cl, —Br, or —I groups.

The term “hydrogel” as used herein refers to a gel in which the swelling agent is water. The term “gel” refers to a non-fluid colloidal network or polymer network that is expanded through its volume by a fluid. The term “swelling agent” is a fluid used to swell a gel or network.

The term “hydroperoxide” refers to —O—O—H groups.

The term “hydroxyl” (or “hydroxy”) refers to —OH groups.

The term “hydroxyl protecting group” (or “hydroxy protecting group”) signifies any group commonly used for the temporary protection of an —OH group, including but not limited to, alkoxycarbonyl, acyl, aroyl, alkylsilyl or alkylarylsilyl groups (hereinafter referred to simply as “silyl” groups), alkoxyalkyl, and arylmethyl groups. Alkoxycarbonyl protecting groups are alkyl-O—C(O)— groups such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. Alkoxyalkyl protecting groups are groups such as methoxymethyl, ethoxymethyl, methoxyethoxymethyl, or tetrahydrofuranyl and tetrahydropyranyl. Arylmethyl groups are groups such as benzyl and p-methoxybenzyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. Where multiple —OH groups are present, such groups may be protected as cyclic ethers, such as 1,3-dioxolanes and 1,3-dioxanes (e.g., acetonides). An extensive list of protecting groups for —OH groups may be found in Protective Groups in Organic Synthesis, Greene, T. W.; Wuts, P. G. M., John Wiley & Sons, New York, N.Y., (3rd Edition, 1999), which is hereby incorporated by reference in its entirety and for all purposes as if fully set forth herein.

The term “leaving group” or “LG” refers to groups readily displaceable by a nucleophile, such as an amine, alcohol, phosphorus, or thiol nucleophile or their respective anions. Such leaving groups are well known and include carboxylates, N-hydroxysuccinimide, N-hydroxybenzotriazole, halogen (halides), triflates, tosylates, mesylates, alkoxy, thioalkoxy, phosphinates, phosphonates and the like. In addition, the term “leaving group” or “LG” is meant to encompass leaving group precursors (i.e., moieties that can easily be converted to a leaving group upon simply synthetic procedures such as alkylation, oxidation or protonation). Such leaving group precursors and methods for converting them to leaving groups are well known to those of ordinary skill in the art.

The term “peroxide” refers to —O—O— groups.

A “protected hydroxyl” group is an —OH group protected with a hydroxyl protecting group.

The term “pyranose” as used herein refers to carbohydrates and substituted derivatives of carbohydrates that include a tetrahydropyran ring. Non-limiting examples of pyranoses include, but are not limited to, glucose, galactose, idose, and the like.

The term “thio” (or “thioether” or sulfide) refer to —S— moieties, bonded to carbon atoms of other organic moieties.

The term “thiol” refers to —SH moieties.

The hydrogels of the present technology may be prepared by polymerization of one or more acrylamide-functionalized monomers. Such acrylamide-functionalized monomers may be prepared through the functionalization of aminomethyl-substituted tetrahydropyran or aminomethyl-substituted tetrahydrofuran derivatives and salt forms thereof. In some embodiments, the aminomethyl-substituted tetrahydropyran or tetrahydrofuran derivatives have the Formula III as shown below:

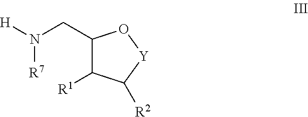

where Y is —C(H)(R³)— or —C(H)(R³)—C(H)(R⁴)—; R¹, R², R³, and R⁴ are independently an —OH, a protected hydroxyl group, or an —NHR⁸, where R⁸ is hydrogen, a methyl group, or an ethyl group, provided that not more than one of R¹, R², R³, and R⁴ is an —NHR⁸, and R⁷ is hydrogen, a methyl group, or an ethyl group. Thus, the compounds of Formula III may be monoamino compounds or bis(amino) compounds. The compounds of Formula III may include protected hydroxyl groups, including but not limited to, silyloxy groups, esters, ethers, and the like. In this regard, the substituents R¹, R², R³, and R⁴ may independently be groups such as TMSO—, TBDMSO—, TBDPSO—, CH₃C(O)—, CF₃C(O)O—, CH₃OCH₂O—, benzoyloxy, THPO— and the like. Furthermore, any two hydroxyl groups may be protected in the form of ring, such as a 1,3-dioxolane, 1,3-dioxane, 1,32-dioxasilolane, or 1,3,2-dioxasilinane ring using reagents commonly known in the art (e.g., acetone, 2-methoxypropene, 2,2-dimethoxypropane, dihalodialkylsilanes, etc.). Some compounds of Formula III are commercially available, such as 6-amino-6-deoxy-D-glucose hydrochloride (BOC Sciences, Shirey, N.Y.). Compounds of Formula III may also be prepared by known methods, such as from aldohexose or aldopentose sugars. Examples of such preparations include those disclosed by Keisuke, K. et al. in Makromolekulare Chemie (1986), 187, 1359-65, which is hereby incorporated by reference in its entirety and for all purposes as if fully set forth herein. Some examples of aldohexose and aldopentose sugars are shown below, as D-isomers, in pyranose and furanose form, respectively.

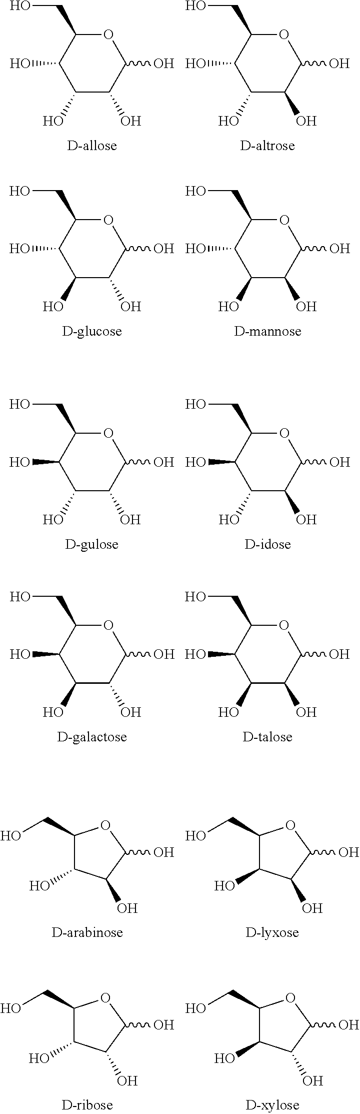

The present technology contemplates the use of any stereoisomers of the compounds of Formula III. Further, there is no requirement that the compounds of Formula III be prepared from aldohexose or aldopentose sugars, such as those shown above. However, because of the ready availability of certain naturally-occurring sugars (e.g., D-glucose or D-galactose), it may be desired to prepare compounds of Formula III from such sugars simply for reasons of cost. Particularly useful compounds of Formula III include 6-(aminomethyl)tetrahydro-2H-pyran-2,3,4,5-tetraol (Formula IIIA) and 3-amino-6-(aminomethyl)tetrahydro-2H-pyran-2,4,5-triol (Formula IIIB) the structures of which are shown below.

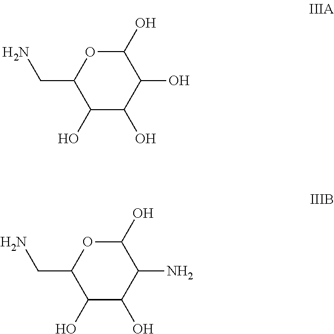 [SMILES: NCC1OC(O)C(O)C(O)C1O ; NCC1OC(O)C(N)C(O)C1O]

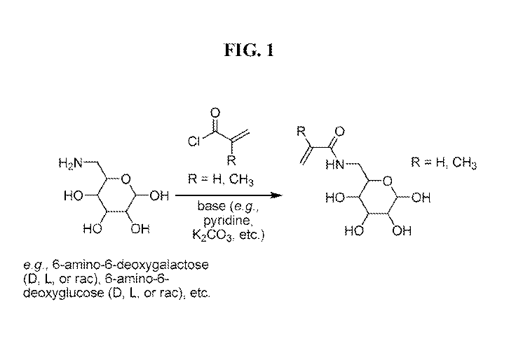

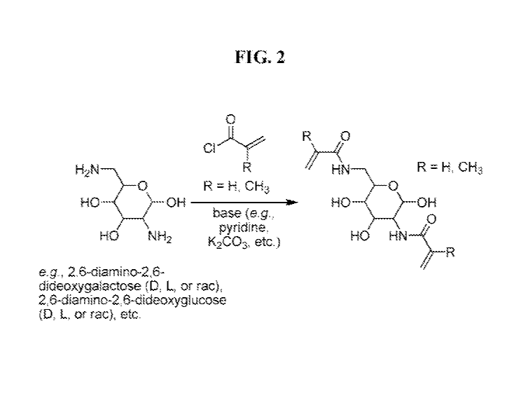

The compound of Formula IIIA embraces such compounds as 6-amino-6-deoxy-D-glucose and 6-amino-6-deoxy-D-galactose, the use of which are described in Examples 1-3 and illustrated in FIG. 1. The compound of Formula IIIB embraces such compounds as 2,6-diamino-2,6-deoxy-D-glucose, and 2,6-diamino-2,6-dideoxy-D-galactose, the use of which are described in Examples 4-5 and illustrated in FIG. 2.

According to one aspect, the compound of Formula III is functionalized to provide a compound of Formula I, shown below:

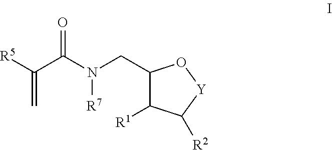

and stereoisomers thereof, where Y is —C(H)(R³)— or —C(H)R³)—C(H)(R⁴)—; R¹, R², R³, and R⁴ are independently an —OH, a protected hydroxyl group, or a group of Formula II:

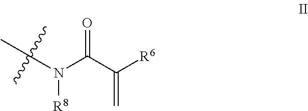

provided that not more than one of R¹, R², R³, and R⁴ is a group of Formula II; R⁵ and R⁶ are independently selected from hydrogen or a methyl group; and R⁷ and R⁸ are independently hydrogen, a methyl group, or an ethyl group. Thus, the compound of Formula I includes at least one acrylamide moiety.

The preparation of the compound of Formula I will typically be accomplished by contacting a compound of Formula III with an acrylate reagent, optionally in the presence of base. Acrylate reagents include, but are not limited to reagents such as acryloyl chloride and methacryloyl chloride. The amount of acrylate reagent used will generally be a stoichiometric amount, or a slight excess (such as about 1%, about 2%, about 3%, about 4%, about 5%, about 10%, about 25%, about 50%, or more than 50% excess), based upon the number of amino groups in the compound of Formula III. Typically, the reaction of the compound of Formula III with the acrylate reagent proceeds in good yield, with good levels of chemoselectivity, i.e., the more reactive amino groups are selectively functionalized in the presence of the hydroxyl groups, as to provide an acrylamide rather than an acrylate ester. In this regard, the functionalization reaction may proceed without the need for hydroxyl protecting groups, i.e., the hydroxyl groups of the compound of Formula III need not be protected. Where the hydroxyl groups of the compound of Formula III are protected, any suitable hydroxyl protecting groups may be used, including but not limited to, trimethylsilyl (TMS), t-butyldimethylsilyl (TBDMS), acetyl, benzyl (Bn), benzoyl (Bz), or methoxymethyl (MOM) protecting groups.

The functionalization of the compound of Formula III with the acrylate reagent optionally occurs in the presence of base. Non-aqueous reaction systems will typically employ a base, while biphasic aqueous reaction systems may not. As will be appreciated by those of skill in the art, a variety of bases may be used, including organic bases and inorganic bases. Examples of suitable organic bases include, but are not limited to pyridine, dimethylaminopyridine, triethylamine, and/or diisopropylethylamine. In the case of inorganic bases, it is generally preferred to select a base which will react with byproducts formed in the reaction but which will not react with the acrylate reagent (e.g., HCl is a byproduct when acryloyl or methacryloyl chloride are used). Examples of suitable inorganic bases include, but are not limited to alkali metal hydroxides (e.g., sodium hydroxide, potassium hydroxide), alkali metal carbonates (e.g. sodium carbonate, potassium carbonate, cesium carbonate), alkaline earth metal carbonates (e.g., calcium carbonate, magnesium carbonate), and the like.

As will be appreciated by those of skill in the art, where a given compound of Formula III is available in salt form (including, but not limited to, a hydrochloride salt, hydrobromide salt, a sulfate or hydrogen sulfate salt), such a salt form may be neutralized or free-based with an organic or inorganic base prior to functionalization reaction with the acrylate reagent. In this regard, the base used to neutralize the salt may be the same or different from the optional base used in the functionalization of the compound of Formula III with the acrylate reagent.

A variety of classes of reaction solvents may be employed in the preparation of the compound of Formula I, including, but not limited to, water, alcohols, ethers, glycol ethers, ketones, amides, nitriles, hydrocarbons, halogenated hydrocarbons, or mixtures of any two or more thereof. The reaction solvent may include, but is not limited to, water, methanol, ethanol, n-propanol, isopropanol, n-butanol, tert-butanol, diethyl ether, diisopropyl ether, dibutyl ether, tetrahydrofuran, 2-methyltetrahydrofuran, monoglyme, diglyme, acetone, 2-butanone, N,N-dimethylformamide, N,N-dimethylacetamide, N-methylpyrrolidinone, acetonitrile, hexane, toluene, xylenes, dichloromethane or chloroform.

The reaction of the compound of Formula III with the acrylate reagent and optional base and solvent(s) are performed at a temperature and for a time period sufficient to produce the compound of Formula I. In some embodiments, the reaction is performed at a temperature of about −30° C., about −20° C., about −10° C., about 0° C., about 10° C., about 20° C., or about 25° C., or ranges between any two of these values. In other embodiments, the reaction may be performed with heating. In this regard, the reaction may be performed at a temperature above room temperature, up to and including a refluxing temperature of the reaction mixture. The reaction may be performed at a temperature of about 25° C., about 30° C., about 40° C., about 50° C., about 60° C., about 70° C., about 80° C., about 90° C., about 100° C., about 120° C., about 140° C., about 160° C., about 180° C., about 200° C., about 220° C., about 240° C., about 260° C., about 280° C., or about 300° C., or ranges between any two of these values. The reaction may be performed for about 10 minutes to about 10 hours, about 30 minutes to about 10 hours, about 30 minutes to about 6 hours, about 10 minutes to about 8 hours, about 30 minutes to about 8 hours, about 1 hour to about 8 hours, about 3 hours to about 8 hours, about 4 hours to about 6 hours, or about 5 hours. Specific examples of the reaction time include about 10 minutes, about 30 minutes, about 1 hour, about 2 hours, about 4 hours, about 6 hours, about 8 hours, about 10 hours, about 20 hours, about 30 hours, and ranges between any two of these values. The compound of Formula I thus prepared may optionally be purified by any number of methods known in the art, including but not limited to, chromatography, distillation, filtration, recrystallization, and the like. Alternatively, the compound of Formula I may be used directly in subsequent reaction without any substantial purification. The preparation of compounds with the Formula I are described in the Examples 1-5 and further illustrated in FIGS. 1-2.

The compound of Formula I can include one or two acrylamide groups. In this regard, the compound of Formula I may be used as a monomer to prepare hydrogels which include repeating units derived from the compound of Formula I. The compound of Formula I, or multiple compounds of Formula I may be polymerized with other monomers. For example, a single compound of Formula I may be polymerized in the absence of other monomers to give a homopolymeric polyacrylamide hydrogel. Multiple monomers with Formula I may be copolymerized to provide a polyacrylamide hydrogel which is a copolymer. Furthermore, where the one or more compounds of Formula I include two acrylamide groups, the hydrogel obtained upon polymerization may be a cross-linked hydrogel. In this aspect, the technology allows for the preparation of cross-linked acrylamide hydrogels without the use of potentially toxic cross-linking agents. In one embodiment, the hydrogel includes repeating units from compounds of Formula IA, IB, or both IA and IB, which are shown below:

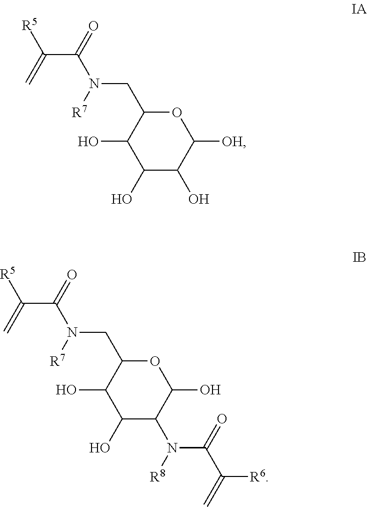

Where the hydrogel includes repeating units from the compounds of Formula IA and Formula IB, the level of cross-linking in the hydrogel will depend on the amount of repeating units of the compound of Formula IB. The hydrogel may be: a lightly cross-linked, a highly swellable polymeric material; a heavily cross-linked, rigid, and minimally swellable polymeric material; or a polymeric material with intermediate properties. The hydrogel may contain about 0.01 wt %, about 0.05 wt %, about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, or a range between any two of these values of the compound of Formula IB based on the total weight of the hydrogel. In one embodiment, the hydrogel includes from about 90 wt % to about 99.9 wt % of the compound of Formula IA and from about 0.1 wt % to about 10 wt % of the compound of Formula IB, based on the total weight of the compounds of Formula IA and IB.

For clarity, hydrogels of the present technology may be prepared which do not include repeating units derived from the compound of Formula IB, i.e., the hydrogels may not be cross-linked. For example, polymerization of the compound of Formula IA may provide a high-molecular weight and water-swellable hydrogel which is not cross-linked. Furthermore, hydrogels of the present technology may also be cross-linked with compounds not of Formula IB. For example, a hydrogel which includes repeating units derived from the compound of Formula IA may be cross-linked with a variety of cross-linkers known in the art, including but not limited to, diisocyanates (e.g., cyclohexane diisocyanate), dicarboxylic acids (e.g., sebacic acid), divinyl benzene, di- or tri-acrylates (e.g., ethylene glycol dimethacrylate, polyethylene glycol dimethacrylate, pentaerythritol triacrylate), and the like.

As indicated above, one or more compounds with Formula I may be copolymerized with other monomers to provide a polyacrylamide, a polyacrylate, a polyvinyl, an epoxy, a polyurea, a polyurethane, an alkyd resin, or a copolymer of any of the foregoing. For example, a compound or multiple compounds of Formula I may be copolymerized with an acrylate monomer (e.g., methyl methacrylate, acrylic acid, methacrylic acid, hydroxyethyl methacrylic acid, hydroxypropyl methacrylic acid, and the like), a vinyl monomer (e.g., butadiene, vinyl chloride, vinyl acetate, and the like), a stryrenic monomer (e.g., styrene), an epoxy-containing monomer, and the like.

Hydrogels of the present technology may be prepared by polymerizing one or more compounds of Formula I or stereoisomers thereof in an aqueous solution to form the hydrogel. Alternatively, the hydrogels of the present technology can be prepared by polymerizing one or more compounds of Formula I in a non-aqueous solution (in which the compounds are soluble) to form a polymer which may subsequently swelled with water to form the present hydrogels. Where the polymerization is performed in an aqueous solution, the aqueous solution may include other solvents in addition to water, including but not limited to alcohols, ethers, glycol ethers, glycols, ketones, amides, nitriles, hydrocarbons, halogenated hydrocarbons, or mixtures of any two or more thereof. Thus, in addition to water, the solvent may include, methanol, ethanol, n-propanol, isopropanol, n-butanol, tert-butanol, propylene glycol, diethyl ether, diisopropyl ether, dibutyl ether, tetrahydrofuran, 2-methyltetrahydrofuran, monoglyme, diglyme, acetone, 2-butanone, N,N-dimethylformamide, N,N-dimethylacetamide, N-methylpyrrolidinone, acetonitrile, hexane, cyclohexane, toluene, xylenes, dichloromethane or chloroform. As will be appreciated by those of skill in the art, where a given solvent is otherwise wholly or partly immiscible with water, one or more surfactants may optionally be added. In one embodiment, the polymerization is performed in water or phosphate-buffered saline (PBS). In another embodiment, the polymerization is performed in aqueous tetrahydrofuran. In another embodiment, the polymerization is performed in a mixture of cyclohexane and water in the presence of the surfactant sorbitan monooleate.

Typically, the polymerization of the compound of Formula I will be initiated with the aid of an initiator, the selection of which will be determined based upon the identity of monomers used and the desired physical properties of the resultant hydrogel (e.g., molecular weight, viscosity, etc.). As used herein, the term “initiator” means a compound that includes at least one site from which a polymerization reaction can be initiated. The initiator may be a thermal initiator or a photochemical initiator. Examples of suitable radical initiators include, but are not limited to: azo compounds (including, but not limited to, azobisisobutyronitrile (AIBN), azobiscyclohexanecarbonitrile (ABCN), and 2,2′-azobis 4-methoxy-2,4-dimethylvaleronitrile); acyl peroxides (including, but not limited to, lauroyl peroxide); aroyl peroxides (including, but not limited to, benzoyl peroxide); alkyl peroxides (including, but not limited to, tert-butyl peroxide and dicumyl peroxide); alkyl hydroperoxides (including, but not limited to, cumene hydroperoxide), persulfates (including, but not limited to, ammonium persulfate, sodium persulfate, and potassium persulfate), and the like. In some embodiments, the initiator is a thermal initiator such as ammonium persulfate or AIBN, or is a photochemical initiator such as DAROCUR 1173 (2-hydroxy-2-methyl-1-phenylpropan-1-one, available from BASF). As will be appreciated by those skilled in the art, the polymerization reaction may also be initiated with a redox couple, such as a persulfate in the presence of either sodium metabisulfate or sodium thiosulfate.

The polymerization reaction may be performed at a temperature below room temperature, about room temperature, about 30° C., about 40° C., about 50° C., about 60° C., about 70° C., about 80° C., about 90° C., about 100° C., about 120° C., about 140° C., about 160° C., about 180° C., about 200° C., about 220° C., about 240° C., about 260° C., about 280° C., or about 300° C., or ranges between any two of these values. In one embodiment, the polymerization reaction is performed at a temperature of about room temperature. In another embodiment, the polymerization reaction is performed at about 40° C.

The hydrogels provided by the present technology described herein may optionally be purified by methods known in the art. Such methods include, but are not limited to, purification by precipitation of the hydrogel from a solution of the polymer and/or by size exclusion chromatography of the polymers comprising the hydrogel. Furthermore, excess solvents, reactants, and by-products may be removed by washing with water, acetone or other suitable solvents and/or subjecting the hydrogel to reduced vacuum, spray drying and/or by lypophilization.

The hydrogels of the present technology may include from 2 to about 1,000,000 repeating units derived from the compound of Formula I. The hydrogels may include 2 repeating units, about 5 repeating units, about 10 repeating units, about 50 repeating units, about 100 repeating units, about 500 repeating units, about 1,000 repeating units, about 4,000 repeating units, about 5,000 repeating units, about 10,000 repeating units, about 40,000 repeating units, about 50,000 repeating units, about 100,000 repeating units, about 200,000 repeating units, about 300,000 repeating units, about 400,000 repeating units, about 500,000 repeating units, about 600,000 repeating units, about 700,000 repeating units, about 800,000 repeating units, about 900,000 repeating units, about 1,000,000 repeating units derived from the compound of Formula I, or a range between and including any two of these values. In a certain embodiment, the hydrogel includes from about 4,000 to about 40,000 repeating units derived from the compound of Formula I.

The hydrogels of the present technology may have a molecular weight from about 500 to about 250,000,000 Daltons. In some embodiments, the hydrogels have a molecular weight of about 500 Daltons, about 1,250 Daltons, about 2,500 Daltons, about 12,500 Daltons, about 25,000 Daltons, about 125,000 Daltons, about 250,000 Daltons, about 1,250,000 Daltons, about 2,500,000 Daltons, about 10,000,000 Daltons, about 12,500,000 Daltons, about 25,000,000 Daltons, about 50,000,000 Daltons, about 75,000,000 Daltons, about 100,000,000 Daltons, about, 125,000,000 Daltons, about 150,000,000 Daltons, about 175,000,000 Daltons, about 200,000,000 Daltons, about 225,000,000 Daltons, about 250,000,000 Daltons, or a range between and including any two of these values. In a certain embodiment, the hydrogel has a molecular weight from about 1,000,000 Daltons to about 10,000,000 Daltons. In another embodiment, the hydrogel has a molecular weight from about 700 Daltons to about 1,000,000 Daltons.

Any of the hydrogels described herein may entrain or otherwise include biomolecules such as collagen, one or more drugs, and/or cells. Further, any of the hydrogels (with or without collagen, one or more drugs, and/or cells) may further be implanted into a patient in need thereof, such as in a human or other animal patient. Examples of an aminal patient include, but are not limited to, a mammal patient such as a dog, cat, rabbit, rodent (e.g., a rat or a mouse), pig, cow, horse, sheep, goat, or elephant. In this regard, the hydrogels will find applications in a number of biomedical fields including wound healing, tissue engineering and regeneration, artificial organ growth, and cartilage repair.

Thus, in another aspect, the present technology provides a method of wound healing or tissue regeneration comprising implanting any of the present hydrogels into a patient in need thereof. As used herein, the term “wound” is any disruption, from whatever cause, of normal anatomy of the patient, and further includes any damage, injury, or trauma to tissue, whether internal or external to the patient. The term “tissue” as used herein refers to a group of cells that perform a similar function. Examples include, but are not limited to, a connective tissue, brain tissue, neuronal tissue, retina, skin tissue, hepatic tissue, pancreatic tissue, blood tissue, muscle tissue, cardiac tissue, vascular tissue, renal tissue, pulmonary tissue, gonadal tissue, hematopoietic tissue. In some embodiments of the present technology, the tissue is a connective tissue, including but not limited to, bone tissue (e.g., osseous tissue), loose connective tissue, an extracellular matrix, tendon tissue, ligament tissue, cartilage tissue, annulus fibrosus, and nucleus pulposus. Such a disruption from normal anatomy or damage to tissue may result from traumatic injuries such as mechanical (e.g., physical), thermal, and incisional injuries or elective injuries such as surgery. Wounds may be acute wounds, chronic wounds, infected wounds, sterile wounds, or wounds associated with any disease state. A wound is dynamic and the process of healing is a continuum requiring a series of integrated and interrelated cellular processes that begin at the time of wounding and proceeds beyond the initial wound closure through arrival at a stable scar. The cellular processes are mediated or modulated by humoral substances including but not limited to cytokines, lymphokines, growth factors, and hormones. The term “wound healing” broadly refers to all of the steps involved in the healing of wounds. The term further includes improving, by some form of intervention of the natural cellular processes and humoral substances such that healing is faster, and/or the resulting healed area has less scaring and/or the wounded area possesses tissue tensile strength that is closer to that of uninjured tissue. The term “wound healing” further includes, but is not limited to, any or all of the following processes: granulation, neovascularization, fibrolast, endothelial and epithelial cell migration, extracellular matrix deposition, re-epithelialization, and remodeling. As used therein, the term “issue regeneration” includes but is not limited to the renewal, restoration, or growth of tissue.

The term “implanting,” as used herein, refers to placing or administering any of the hydrogels of the present technology in a desired location within the patient. Thus, the hydrogels can be implanted directly in the patient in vivo for tissue regeneration and/or wound healing. In some embodiments, the hydrogels are implanted near, at, on, or underneath the wound or site of desired tissue regeneration. Methods of implanting polymeric materials such as the present hydrogels are known in the art. For example, the hydrogels can be implanted subcutaneously, intradermally, or into any body cavity. The hydrogels can be implanted by injection, using a suitable delivery means such as by a catheter or a cannula. According to some embodiments of the present technology, the tissue is implanted at a site where it is desired to heal a wound or regenerate tissue.

Where the hydrogels of the present technology include collagen, the collagen may be any of the numerous types of collagen known in the art, for example, Type I collagen. Type, II collagen, Type III collagen, etc. or mixtures of one or more thereof. While not required, it is generally preferred that the antigenic telopeptide portion of the collagen has been removed or substantially reduced. Methods for removing the antigenic telopeptide portion of the collagen are well known in the art. For example, insoluble collagen extracted from various animals may be subjected to alkaline treatment, or may be treated with enzymes such as pepsin, trypsin, chymotrypsin, papain, or pronase to remove the antigenic telopeptide portion. There are no restrictions on the origin of the collagen, and typically collagen obtained from the skin, bone, cartilage, tendon, organs, etc. of birds or mammals such as cows, pigs, rabbits, sheep, or mice may be used. Collagen may be incorporated into the present hydrogels via a number of methods, such as by preparing the polymerizing one or more compounds of Formula I in the presence collagen, as described in Examples 7 and 9. The collagen may also be mechanically mixed or otherwise dispersed in the hydrogel, after the hydrogel has been prepared. The collagen may be covalently bound to the hydrogel (e.g. such as through cross-linking) or reversibly or irreversibly entrained within the hydrogel.

The hydrogel may also include one or more drugs entrained within the hydrogel. Such drugs may be one or more of an anticancer agent, an antiproliferative agent, an antimitotic, an antineoplastic, an anticancer agent, an anti-inflammatory drug, an antiplatelet, an anticoagulant, an antifibrin, an antithrombin, a cytostatic agent, an antibacterial, an antiviral, an antibiotic, an anti-allergic agent, an anti-enzymatic agent, an angiogenic agent, a cyto-protective agent, a cardioprotective agent, a proliferative agent, an ABC A1 agonist, an antioxidant, a growth factor, a cytokine, a protein or peptide, or other drug.

Examples of antiproliferative drugs, antimitotics, antineoplastics, and anticancer agents include, but are not limited to, actinomycins, taxol, docetaxel, paclitaxel, rapamycin, 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin, everolimus, biolimus, perfenidone, methotrexate, azathioprine, vincristine, vinblastine, fluorouracil, doxorubicin hydrochloride, and mitomycin, and derivatives, analogs, prodrugs, and co-drugs thereof.

Examples of anti-inflammatory drugs include, but are not limited to, both steroidal and non-steroidal (NSAID) anti-inflammatory drugs such as clobetasol, alclofenac, alclometasone dipropionate, algestone acetonide, α-amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarimn chloride, seclazone, sennetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, trifltnidate, zidometacin, zomepirac sodium, aspirin (acetylsalicylic acid), salicylic acid, corticosteroids, glucocorticoids, tacrolimus, pimecrolimus and derivatives, analogs, prodrugs, and co-drugs thereof.

Examples of antiplatelet, anticoagulant, antifibrin, and antithrombin drugs include, but are not limited to, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax, calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic, 4-amino-2,2,6,6-tetramethylpiperidine-1-oxyl (4-amino-TEMPO) and derivatives, analogs, prodrugs, and co-drugs thereof.

Examples of cytostatic or antiproliferative agents include, but are not limited to, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Examples of antibacterials include, but are not limited to macrolides or ketolides such as erythromycin, azithromycin, clarithromycin and telithromycin; β-lactams including penicillin, cephalosporin, and carbapenems such as carbapenem, imipenem, and meropenem; monobactams such as penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomycin, kanamycin, paromycin, gentamicin, tobramycin, amikacin, netilmicin, spectmomycin, sisomicin, dibekalin and iseparmicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin, colymycin, trimethoprim, and bacitracin.

Examples ofantibacterials include, but are not limited to, anti-HIV agents (e.g., CCR5 antagonist, CXCR4 antagonist, reverse transcriptase inhibitors, fusion inhibitors), anti-influenza viral agents (e.g., oseltamivir phosphate, zanamivir hydrate), anti-herpes virus agents (e.g., acyclovir), interferon-α or β, and various types of immunoglobulins

Examples of anti-allergic agents include, but are not limited to, pemirolast potassium and histamine H1 antagonists (e.g., loratidine, benadryl, antazoline, pheniramine, chlorpheniramine, and the like),

Examples of growth factors and/or cytokines include, but are not limited to, interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Cytokines may be B/T-cell differentiation factors, BIT-cell growth factors, mitogenic cytokines, chemotactic cytokines, colony stimulating factors, angiogenesis factors, IFNα, IFN-β, IFN-γ, IL1, IL2, IL3, IL4, IL5, IL6, IL7, IL8, IL9, IL10, IL11, IL12, IL13, IL14, IL15, IL16, IL17, IL18, etc., leptin, myostatin, macrophage stimulating protein, platelet-derived growth factor, TNF-α, TNF-β, NGF, CD40L, CD137L/4-IBBL, human lymphotoxin-.beta., G-CSF, M-CSF, GM-CSF, PDGF, IL-1α, IL-1-β, IP-10, PF4, GRO, 9E3, erythropoietin, endostatin, angiostatin, VEGF or any fragments or combinations thereof. Other cytokines include members of the transforming growth factor (TGF) supergene family include the beta transforming growth factors (for example TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB). Growth factors may be isolated from native or natural sources, such as from mammalian cells, or may be prepared synthetically, such as by recombinant DNA techniques or by various chemical processes. In addition, analogs, fragments, or derivatives of these factors may be used, provided that they exhibit at least some of the biological activity of the native molecule. For example, analogs may be prepared by expression of genes altered by site-specific mutagenesis or other genetic engineering techniques.

Examples of peptides and proteins which may be included in the present hydrogels include, but are not limited to, phosphorothioate deoxyribo-oligonucleotide, antisense oligonucleotides, tissue plasminogen activator, fibrin, fibrinogen, fibronectin, thrombin, anti-thrombin III, trypsin, zymogen, angiotensinogen, trypsinogen, chymotrypsinogen, pepsinogen, osteoids, vitronectin, laminin, gelin-S, Cdx1 protein, Cdx2 protein, Cdx4 protein, HSP60, HSP70, HSP90, HSP110, trithorax-group proteins, polycomb-group proteins, myosin, kinesin, dynein, G proteins, isoleucine-proline-proline and valine-proline-proline, glutathione, tryptone, C-type natriuretic peptides, defensin, α-defensin, β-defensin, θ-defensin, defensin-like peptides, retrocyclin, peptones, actinomycin, bacitracin, calcium dependent antibiotic, daptomycin, vancomycin, tyrocidine, gramicidin, thiostrepton, zwittermicin A, ACV-tripeptide, epothilone, bleomycin, cicloporin, enterobactin, myxochelin A, indigoidine, microcystins, nodularins, cyanotoxins, cyanophycin, HC-toxin, AM-toxin, victorin, cyclosporine A, and derivatives, analogs, prodrugs, and codrugs thereof.

Examples of other drugs which may be included in the hydrogel include, but are not limited to, α-interferon, genetically engineered epithelial cells, dexamethasone, antisense molecules which bind to complementary DNA to inhibit transcription, and ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy, analgesics and analgesic combinations; anorexics; antihelmintics; antiarthritics, antiasthmatic agents; anticonvulsants; antidepressants; antidiuretic agents; antidiarrheals; antihistamines; antimigraine preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and derivatives, analogs, prodrugs, and codrugs thereof.

In some embodiments, the hydrogel includes one or more drugs selected from anticancer agents, anti-inflammatory drugs, antibacterials, antivirals, or growth factors. As shown in Example 8, hydrogels including an entrained anticancer drug may be prepared by aqueous polymerization of compounds of Formula I in the presence of the anticancer drug. The one or more drugs may also be added to the hydrogel after the preparation of the hydrogel, such as by mechanical mixing or other method of dispersing the drug within the hydrogel. Such hydrogels including drugs may further be implanted into a patient in need thereof. In this regard, the present technology also provides a method of releasing a drug in a sustained fashion from the hydrogels described herein. The term “sustained fashion” refers to the release of the drug from the hydrogel over a prolonged period of time. When such drug-containing hydrogels are implanted into a patient, the drug may be released in a sustained fashion such that the drug becomes available for bio-absorption in the patient Where one or more drugs are entrained in the hydrogel and such a hydrogel is implanted into a patient, the hydrogel can release the drug in a sustained fashion over a period of about 1 day, about 1 week, about 1 month, about 3 months, about 6 months, about 9 months, about 1 year, about 2 years, about 3 years, or longer than 3 years. The release periods of the drug may be adjusted using techniques commonly known in the art, such as through variation of the level of cross-linking within the hydrogel and appropriate selection of monomers used to prepare the hydrogel.

The present hydrogels may be used as scaffolds to support living cells and/or living tissue. In this regard the hydrogels the present hydrogels will find use as cell culture media or use in other cell culture applications. Cells or living tissues may be entrained within the hydrogel. As used herein, the term “entrained” means that the cells or living tissues are integrated, attached, or in contact with the hydrogel. Any cell compatible with the present hydrogels may be used, including, but not limited to, stem cells, differentiated cells, or cells of any of the tissues described herein. The cells may be derived from of a variety of cell lines, including but not limited to those indicated in Table 1 or Table 2.

TABLE 1
Cell lines from various tissues and organisms

| Cell line | Organism | Origin tissue |
|---|---|---|
| 293-T | Human | Kidney (embryonic) |
| 3T3 cells | Mouse | Embryonic fibroblast |
| 721 | Human | Melanoma |
| 9L | Rat | Glioblastoma |
| A2780 | Human | Ovary |
| A2780ADR | Human | Ovary |
| A2780cis | Human | Ovary |
| A172 | Human | glioblastoma |
| A20 | Murine | B lymphoma |
| A253 | Human | Head and neck carcinoma |
| A431 | Human | Skin epithelium |
| A-549 | Human | Lungcarcinoma |
| ALC | Murine | bone marrow |
| B16 | Murine | Melanoma |
| B35 | Rat | Neuroblastoma |
| BCP-1 cells | Human | PBMC |
| BEAS-2B | Human | Lung |
| bEnd.3 | Mouse | Brain/Cerebral cortex |
| BHK-21 | Hamster | Kidney |
| BR 293 | Human | Breast |
| BxPC3 | Human | pancreatic adenocarcinoma |
| C3H-10T1/2 | Mouse | Embryonic mesenchymal cell |
| | | line |
| C6/36 | Asian tiger | larval tissue |
| | mosquito | |
| Cal-27 | Human | Tongue |
| CHO | hamster | Ovary |
| COR-L23 | Human | Lung |
| COR-L23/CPR | Human | Lung |
| COR-L23/5010 | Human | Lung |
| COR-L23/R23 | Human | Lung |
| COS-7 | Ape - | Kidney |
| | Cercopithecus | |
| | aethiops | |
| | (Chlorocebus) | |
| COV-434 | Human | Ovary |
| CML T1 | Human | CML acute phase |
| CMT | Dog | Mammary gland |
| CT26 | Murine | Colorectal Carcinoma |
| D17 | canine | osteosarcoma |
| DH82 | canine | histiocytosis |
| DU145 | Human | Androgen insensitive |
| | | carcinoma |
| DuCaP | Human | Metastatic Prostate Cancer |
| EL4 | Mouse | |
| EM2 | Human | CML blast crisis |
| EM3 | Human | CML blast crisis |
| EMT6/AR1 | Mouse | Breast |
| EMT6/AR10.0 | Mouse | Breast |
| FM3 | Human | Metastatic lymph node |
| H1299 | Human | Lung |
| H69 | Human | Lung |
| HB54 | hybridoma | hybridoma |
| HB55 | hybridoma | hybridoma |
| HCA2 | Human | fibroblast |
| HEK-293 | Human | Kidney (embryonic) |
| HeLa | Human | Cervical cancer |
| Hepa1c1c7 | Mouse | Hepatoma |
| HL-60 | Human | Myeloblast |
| HMEC | Human | |
| HT-29 | Human | Colon epithelium |
| Jurkat | Human | T-Cell-Leukemia |
| JY cells | Human | Lymphoblastoid |
| K562 cells | Human | Lymphoblastoid |
| Ku812 | Human | Lymphoblastoid |
| KCL22 | Human | Lymphoblastoid |
| KG1 | Human | Lymphoblastoid |
| KYO1 | Human | Lymphoblastoid |
| LNCap | Human | prostatic adenocarcinoma |
| Ma-Mel 1, 2, | Human | |
| 3 . . . 48 | | |
| MC-38 | Mouse | |
| MCF-7 | Human | Mammary gland |
| MCF-10A | Human | mammary gland |
| MDA-MB-231 | Human | Breast |
| MDA-MB-468 | Human | Breast |
| MDA-MB-435 | Human | Breast |
| MDCK II | Dog | Kidney |
| MDCK II | Dog | Kidney |
| MOR/0.2R | Human | Lung |
| MONO-MAC 6 | Human | WBC |
| MTD-1A | Mouse | |
| MyEnd | Mouse | |
| NCI-H69/CPR | Human | Lung |
| NCI-H69/LX10 | Human | Lung |
| NCI-H69/LX20 | Human | Lung |
| NCI-H69/LX4 | Human | Lung |
| NIH-3T3 | Mouse | embryo |
| NALM-1 | | peripheral blood |
| NW-145 | | |
| OPCN/OPCT | | |
| cell lines | | |
| Peer | Human | T cell leukemia |
| PNT-1A/PNT 2 | | |
| RenCa | Mouse | |
| RIN-5F | Mouse | Pancreas |
| RMA/RMAS | Mouse | |
| Saos-2 cells | Human | |
| Sf-9 | insect - | Ovary |
| | Spodoptera | |
| | frugiperda | |
| | (moth) | |
| SkBr3 | Human | |
| T2 | Human | |
| T-47D | Human | Mammary gland |
| T84 | Human | colorectal Carcinoma/ |
| | | Lungmetastasis |
| THP1 cell line | Human | Monocyte |
| U373 | Human | Glioblastoma-astrocytoma |
| U87 | Human | glioblastoma-astrocytoma |
| U937 | Human | Leukaemic monocytic |
| | | lymphoma |
| VCaP | Human | Metastatic prostate cancer |
| Vero cells | African Green | Kidney epithelium |
| | Monkey | |
| WM39 | Human | skin |
| WT-49 | Human | Lymphoblastoid |
| X63 | Mouse | Melanoma |
| YAC-1 | Mouse | Lymphoma |
| YAR | Human | B-cell |

TABLE 2
Cancer cell lines

| Origin | Cell Line | Cell Type |
|---|---|---|
| Bone | Saos-2 | Bone; osteosarcoma |
| Bone marrow | KG-1 | Bone marrow |
| Bone marrow | TF-1 | Bone marrow |
| Brain | IMR-32 | Neuroblastoma |
| Brain | KAN-TS | Neuroblastoma |
| Brain | SMS-KCN | Neuroblastoma |
| Brain | SMS-KCNR | Neuroblastoma |
| Brain | H4 | Neuroglioma |
| Brain | U373 | Glioblastoma; astrocytoma; grade |
| | | III |
| Brain | SK-N-SH | Neuroblastoma; metastasis to |
| | | bone marrow |
| Brain | SK-N-MC | Neuroepithelioma; metastasis to |
| | | supra-orbital area |
| Brain | SH-SY5Y | Neuroblstoma; metastatic to |
| | | bone marrow |
| Brain | SK-N-F1 | Neuroblastoma; metastatic to |
| | | bone marrow |
| Breast | MB-468 | Adenocarcinoma |
| Breast | MDA MB 231 | Adenocarcinoma |
| Breast | MDA-MB-361 | Adenocarcinoma |
| Breast | SK-BR-3 | Adenocarcinoma |
| Breast | BT-20 | Carcinoma |
| Breast | Hs-578T | Carcinoma |
| Breast | Hs 578 Bst | Derived from normal breast |
| | | tissue periperal to Hs578T |
| | | carcinoma |
| Breast | MCF-7 | Carcinoma |
| Breast | MT-3 | Carcinoma |
| Breast | BT-549 | Primary ductal carcinoma |
| Breast | HCC-38 | Primary ductal carcinoma |
| Breast | HCC-70 | Primary ductal carcinoma |
| Breast | HCC-1143 | Primary ductal carcinoma |
| Breast | HCC-1187 | Primary ductal carcinoma |
| Breast | HCC-1395 | Primary ductal carcinoma |
| Breast | HCC-1500 | Primary ductal carcinoma |
| Breast | BCC-1599 | Primary ductal carcinoma |
| Breast | HCC-1937 | Primary ductal carcinoma |
| Breast | HCC-2157 | Primary ductal carcinoma |
| Breast | HCC-1569 | Mammary gland; primary |
| | | metaplastic carcinoma |
| Breast | HCC-1806 | Mammary gland; carcinoma; |
| | | primary acantholytic |
| Breast | BT-474 | Mammary gland; ductal |
| | | carcinoma |
| Breast | BT-549 | Mammary gland; ductal |
| | | carcinoma |
| Breast | HCC-202 | Mammary gland; carcinoma; |
| | | primary ductal |
| Breast | HCC-1419 | Mammary gland; carcinoma; |
| | | primary ductal |
| Breast | UACC-893 | Mammary gland; carcinoma; |
| | | primary ductal |
| Breast | HCC-1954 | Mammary gland; ductal |
| | | carcinoma |
| Breast | HCC-2218 | Mammary gland; ductal |
| | | carcinoma |
| Breast | UACC-812 | Mammary gland; ductal |
| | | carcinoma |
| Breast | ZR-75-30 | Mammary gland; ductal |
| | | carcinoma |
| Breast | ZR-75-1 | Mammary gland; breast ductal |
| | | carcinoma |
| Breast | MDA-MB-453 | Mammary gland; pericardial |
| | | effusion |
| Breast | MDA-MB-330 | Mammary gland; pleural |
| | | effusion |
| Breast | MDA-MB-436 | Mammary gland; pleural |
| | | effusion; adenocarcinoma |
| Breast | MDA-MB-468 | Mammary gland; pleural |
| | | effusion; adenocarcinoma |
| Breast | T 47D | Mammary gland; ductal |
| | | carcinoma; pleural effusion |
| Breast | HCC 1008 | Mammary gland; ductal |
| | | carcinoma; stage IIA |
| Breast | MDA-MB-175- | Mammary gland; breast ductal |
| | VII | carcinoma; metastatic |
| Breast | MDA-MB-157 | Mammary gland; breast medulla |
| | | medulallary carcinoma |
| Breast | AU 565 | Carcinoma |
| Breast | BT-483 | Carcinoma |
| Breast | CAL-51 | Carcinoma |
| Breast | CAL-85-1 | Carcinoma |
| Breast | Cama-1 | Carcinoma |
| Breast | COLO 824 | Carcinoma |
| Breast | EFM-19 | Carcinoma |
| Breast | EFM-192A | Carcinoma |
| Breast | EVSA-T | Carcinoma |
| Breast | MDA-MB-415 | Carcinoma |
| Breast | MX-1 | Carcinoma |
| Breast | SUM 52 PE | Carcinoma |
| Breast | SUM 149 PT | Carcinoma |
| Breast | SUM 159 PT | Carcinoma |
| Breast | SUM 185 | Carcinoma |
| Breast | SUM 225 | Carcinoma |
| Cervix | HeLa | Carcinoma |
| Cervix | HeLA HA7 | Carcinoma; HeLa Variant |
| Colon | HT-29 | Adenocarcinoma |
| Colon | CaCo-2 | Colorectal adenocarcinoma |
| Colon | COLO 205 | Colorectal carcinoma |
| Colon | HCT-15 | Colorectal carcinoma |
| Colon | HCT-116 | Colorectal carcinoma |
| Colon | SW-620 | Colorectal carcinoma |
| Colon | T84 | Colorectoal carcinoma; derived |
| | | from metastatic lung site |
| Connective Tissue | HT-1080 | Fibrosarcoma |
| Kidney | A-498 | Carcinoma |
| Kidney | 786-O | Renal cell carcinoma |
| Kidney | ACHN | Renal cell carcinoma |
| Kidney | Caki-1 | Renal cell carcinoma |
| Lung | NCI-H522 | Adenocarcinoma |
| Lung | Calu-3 | Adenocarcinoma, pleural |
| | | effusion |
| Lung | NCI-H23 | Adenocarcinoma; non-small cell |
| | | lung cancer |
| Lung | NCI-H226 | Squamous cell carcinoma; |
| | | mesothelioma |
| Lung | A549 | Carcinoma |
| Lung | NCI-H157 | Carcinoma |
| Lung | NCI-H460 | Large cell lung carcinoma |
| Lung | DMS-53 | Small cell lung carcinoma |
| Lung | DMS-114 | Small cell lung carcinoma |
| Lung | DMS-153 | Small cell lung carcinoma |
| Lung | SHP-77 | Small cell lung carcinoma |
| Muscle | RD | Rhabdomyosarcoma |
| Muscle | Hs 729T | Rhabdomyosarcoma |
| Ovary | NCI/ADR-RES | Ovarian carcinoma |
| Ovary | OVCAR 3 | Ovarian carcinoma |
| Ovary | SK-OV-3 | Ovarian carcinoma |
| Pancreas | CFPAC-1 | Ductal adenocarcinoma; |
| | | metastatic liver site |
| Pancreas | PANC-1 | Carcinoma |
| Peripheral Blood | CCRF-CEM | Acute lymphoblastic leukemia |
| Peripheral Blood | HL-60 | Acute promyelocytic leukemia |
| Peripheral Blood | MOLT-4 | Acute lymphoblastic leukemia |
| Peripheral Blood | THP-1 | Acute monocytic leukemia |
| Peripheral Blood | BDCM | Acute myelogenous leukemia |
| | | (AML) |
| Peripheral Blood | Jurkat | Acute T cell; lymphocyte; |
| | | leukemia |
| Peripheral Blood | K-562 | Chronic myelogenous leukemia |
| | | (CML); bone marrow |
| Peripheral Blood | RPMI-8866 | Chronic myeloid leukemia |
| Peripheral Blood | U-937 | Histiocytic lymphoma |
| Peripheral Blood | SR | Large cell immunoblastic |
| | | lymphoma; pleural effusion |
| Peripheral Blood | RPMI-8226 | Myeloma |
| Prostate | LN CaP | Carcinoma |
| Prostate | PC3 | Adenocarcinoma derived from |
| | | metastatic bone |
| Prostate | DU 145 | Carcinoma derived from |
| | | metastatic brain |
| Skin | A431 | Epidermoid carcinoma |
| Skin | MDA-MB- | Melanoma |
| | 435S | |
| Skin | SK-MEL-2 | Malignant melanoma |
| Skin | SK-MEL-5 | Malignant melanoma |
| Skin | SK-MEL-28 | Malignant melanoma |
| Skin | Malme-3M | Malignant melanoma; lung |
| Skin | A2058 (AZ) | Melama from netastatic lymph |
| | | node |

Any of the hydrogels described herein may be implanted in a patient to support wound healing or tissue regeneration. The hydrogel may also be used to prepare organs or other living tissues for implantation to a patient. For example, the hydrogel (in any form, dry or wetted), can be implanted into a patient as is, or can be seeded with cells prior to implantation in the patient so as to enable proliferation, differentiation, and/or migration of cells in the area of the wound or desired location of tissue regeneration. For example, cells may be cultured on or in the hydrogel, the resultant hydrogel with living cells implanted into the patient. Such living cells may be provided by the patient or may be obtained from another source. As described in Example 9, non-woven, fibrous mats of hydrogels may be prepared through polymerization and electrospinning of aqueous solutions of compounds of Formula I. As understood in the art, electrospinning is a method of producing or drawing fibers from a liquid, using electrical charge. The fibrous mats thus prepared are highly porous and provide a suitable environment for the growth of cells. Such fibrous mats, with or without cells, may then be implanted in a patient to support the creation of new tissue or wound healing.

Hydrogels with pores (cavities) may also be prepared, and such porous hydrogels may further include collagen, one or more drugs, and/or cells. Porous hydrogel scaffolds may be prepared by polymerizing an aqueous solution of one or more compounds of Formula I in the presence of an inert polymer (or other material) which is not soluble under the polymerization conditions. After the hydrogel is formed, the resultant composite (i.e., a composite of the hydrogel and the inert polymer) may be exposed to an appropriate solvent to dissolve or leach the inert polymer from the composite, thereby removing the inert polymer and leaving behind a porous hydrogel with interconnected cavities. As used herein, the term “interconnected cavities” and grammatical forms thereof refers to pores or voids in the hydrogel which are connected to one another and communicate with one another over the hydrogel as a whole, such that a cell or other material can pass from one cavity to another, over the entire hydrogel, and can in theory circulate through all the cavities of the hydrogel. The size and shape of the cavities will be substantially the same as that of the inert polymer. For example, if spherical inert polymer particles are used, the interconnected cavities will be substantially spherical with diameters substantially the same as that of the spherical polymer particles. The inert polymer may be polymer microspheres comprised of an acrylate polymer such as poly(methyl methacrylate). The polymer microspheres may have diameters of about 0.05 μm, about 0.1 μm, about 1 μm, about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, or about 100 μm, or ranges between any two of these values. Microspheres of varying diameters may be used. In some embodiments, the hydrogel includes interconnected cavities with dimensions of about 0.05 μm, about 0.1 μm, about 1 μm about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, or about 100 μm, or ranges between any two of these values. In some such embodiments, the interconnected cavities are substantially spherical and the diameters of the spherical cavities have any of the aforementioned dimensions. In general, the solvent used to extract the inert polymer from the composite material will be selected as to effectively solubilize the inert polymer but not substantially dissolve the hydrogel (or the one or more drugs or collagen, if so included). In some embodiments, the solvent used to extract the composite material is one or more of acetone, dichloromethane, or ethanol.

Porous hydrogel may also be obtained by the preparation of high internal phase emulsion polymers (polyHIPEs) from one or more compounds of Formula I. The term “HIPE” refers to an emulsion in which the dispersed phase (water) takes up a greater volume of the total volume than the continuous phase. On curing by polymerization of the continuous phase, an open-pored polymer forms, which is then, strictly speaking, no longer an emulsion and is often referred to by those of skill in the art as a “polyHIPE” (see for example, Cameron et al. Polymer 2005, 46, 1439-1449, the contents of which is hereby incorporated by reference as if fully set forth herein). The preparation of hydrogels through high internal phase emulsion polymerization is described in Example 9.

It is understood that any of the hydrogels described herein may be dried using methods commonly employed in the art to provide polymeric materials with reduced levels of water or polymeric materials substantially free of water. Likewise, such polymeric materials may be reconstituted or otherwise swelled to hydrogels using water and other aqueous solutions.

The present technology, thus generally described, will be understood more readily by reference to the following examples, which is provided by way of illustration and is not intended to be limiting of the present technology.

### EXAMPLES

### Example 1

### Synthesis of a galactose-derived N-((3,4,5,6-tetrahydroxytetrahydro-2H-pyran-2-yl)methyl)acrylamide

As shown in FIG. 1, an N-((3,4,5,6-tetrahydroxytetrahydro-2H-pyran-2-yl)methyl)acrylamide may be generally synthesized by reaction of a 6-(aminomethyl)tetrahydro-2H-pyran-2,3,4,5-tetraol (or a salt form thereof) with acryloyl chloride in the presence of base. Analogous methacrylamide compounds are prepared using methacryloyl chloride. A galactose-derived N-((3,4,5,6-tetrahydroxytetrahydro-2H-pyran-2-yl)methyl)acrylamide is prepared as follows. Anhydrous tetrahydrofuran (500 mL) is added to a flame dried 1 L three neck flask and is flooded with argon. To the tetrahydrofuran is added 30.0 g (139.5 mmol) of anhydrous 6-amino-6-deoxy-D-galactose hydrochloride and 31.2 g (394.4 mmol) anhydrous pyridine and allowed to dissolve at a temperature of −20° C. Then by drop-wise addition 13.1 g (140.5 mmol) of acryloyl chloride in 50 mL of dry tetrahydrofuran is added to the 6-amino-6-deoxy-D-galactose solution at room temperature followed by warming to 25° C. The reaction is allowed to proceed for five hours at 25° C. The pyridinium chloride is then removed by filtration followed by washing and the solvents are removed by rotary evaporation and high vacuum to yield N-(6-deoxy-D-galactose)acrylamide, a galactose-derived N-((3,4,5,6-tetrahydroxytetrahydro-2H-pyran-2-yl)methyl)acrylamide. The reaction may also be performed using an equivalent molar amount of potassium carbonate as the base, in place of pyridine.

### Example 2

### Alternative Synthesis of a Galactose-Derived N-((3,4,5,6-tetrahydroxytetrahydro-2H-pyran-2-yl)methyl)acrylamide

The synthesis of N-((3,4,5,6-tetrahydroxytetrahydro-2H-pyran-2-yl)methyl)acrylamide may also be accomplished using a biphasic reaction system. Anhydrous tetrahydrofuran (500 mL) is added to a flame dried 1 L three neck flask and flooded with argon. To water, 150 mL at 0° C., is added 30.0 g (139.5 mmol) of anhydrous 6-amino-6-deoxy-D-galactose hydrochloride and is neutralized with and 3.4 g (139.6 mmol) sodium hydroxide and allowed to dissolve. A solution of 13.1 g (140.5 mmol) of acryloyl chloride in 100 mL of methylene chloride is added to the 6-amino-6-deoxy-D-galactose water solution at 0° C. and stirred rapidly to form an emulsion. Sodium hydroxide 1M solution is added dropwise to keep the mixture between a pH of 7-9. The reaction is allowed to proceed for five hours at 0° C. The stirring is then stopped and the phases are allowed to separate. The methylene chloride layer is extracted and the water layer is washed with three 25 mL portions of methylene chloride. The methylene chloride samples are combined, dried, and then the solvent is removed by rotary evaporation and high vacuum to yield N-(6-deoxy-D-galactose)acrylamide.

### Example 3

### Synthesis of a Glucose-Derived N-((3,4,5,6-tetrahydroxytetrahydro-2H-pyran-2-yl)methyl)methacrylamide

Using similar procedures as outlined in Example 1 or Example 2, 6-amino-6-deoxy-D-glucose is reacted with methacryloyl chloride to yield N-(6-deoxy-D-glucose)methacrylamide, a glucose-derived N-((3,4,5,6-tetrahydroxytetrahydro-2H-pyran-2-yl)methyl)methacrylamide.

### Example 4

### Synthesis of a Glucose-Derived N-((3,4,6-trihydroxy-5-methacrylamidotetrahydro-2H-pyran-2-yl)methyl)methacrylamide Cross-Linking Agent

As shown in FIG. 2, an N-((3,4,6-trihydroxy-5-methacrylamidotetrahydro-2H-pyran-2-yl)methyl)methacrylamide is generally synthesized by reaction of a 3-amino-6-(aminomethyl)tetrahydro-2H-pyran-2,4,5-triol (or a salt form thereof) with methacryloyl chloride in the presence of base. Acrylamides are prepared analogously using acryloyl chloride. In particular, a multi-functional cross-linking agent is prepared by the reaction of 2,6-diamino-2,6-dideoxy-D-glucose and methacryloyl chloride in the presence of an organic base. Anhydrous tetrahydrofuran (500 mL) is added to a flame dried 1 L three neck flask and flooded with argon. To the tetrahydrofuran is added 38.6 g (153.7 mmol) of 2,6-diamino-2,6-dideoxy-D-glucose hydrochloride and 32.5 g (411.3 mmol) pyridine and allowed to dissolve. Then by drop-wise addition 33.2 g (317.4 mmol) of methacryloyl chloride in 55 mL of dry tetrahydrofuran is added to the 2,6-diamino-2,6-dideoxy-D-galactose solution at −20° C. followed by warming to room temperature. The reaction is allowed to proceed for five hours at room temperature. Pyridinium chloride is then removed by filtration followed by washing and the solvents are removed by rotary evaporation and high vacuum to yield N,N-(2,6-deoxy-D-glucose)-2,6-methacrylamide, a glucose-derived N-((3,4,6-trihydroxy-5-methacrylamidotetrahydro-2H-pyran-2-yl)methyl)methacrylamide. The reaction may also be performed using an equivalent molar amount of potassium carbonate as the base, in place of pyridine.

### Example 5

### Synthesis of a Galactose-Derived N-((5-acrylamido-3,4,6-trihydroxytetrahydro-2H-pyran-2-yl)methyl)acrylamide Cross-Linking Agent

Using a similar procedure as outlined in Example 4 and shown in FIG. 2 (with either pyridine or potassium carbonate as base), 2,6-diamino-2,6-dideoxy-D-galactose is reacted with acryloyl chloride to yield N,N-(2,6-deoxy-D-galactose)-2,6-acrylamide, a galactose-derived N-((5-acrylamido-3,4,6-trihydroxytetrahydro-2H-pyran-2-yl)methyl)acrylamide.

### Example 6

### Synthesis of Galactose-Derived poly(N-((3,4,5,6-tetrahydroxytetrahydro-2H-pyran-2-yl)methyl)acrylamide

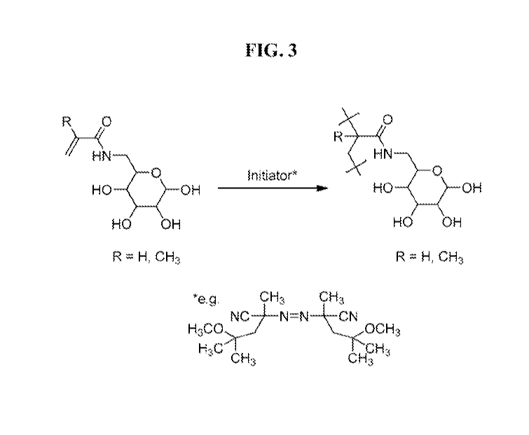

Poly(N-((3,4,5,6-tetrahydroxytetrahydro-2H-pyran-2-yl)methyl)acrylamide is prepared as shown in FIG. 3. A galactose-derived poly(N-((3,4,5,6-tetrahydroxytetrahydro-2H-pyran-2-yl)methyl)acrylamide) may be prepared according to the following procedure. In a four-neck 500 mL separable flask equipped with a mechanical stirrer, a dropping funnel, and a condenser, N-(6-deoxy-D-galactose)acrylamide monomer (Example 1), 20 g; distilled water, 50 mL; and tetrahydrofuran, 150 mL, are added. The flask is warmed to 60° C. under air with stirring, and 0.250 g of 2,2′-azobis(4-methoxy-2,4-dimethylvaleronitrile) is added as a polymerization initiator. The solution changes into a gel-like material in about 20 min. This point is tentatively taken as a standard for the end of reaction. Disappearance of monomer at this point can be monitored by silica gel thin layer chromatography (TLC). After the reaction, the reaction mixture is concentrated under reduced pressure at room temperature, and remaining water in the viscous liquid is removed by freeze-drying to isolate poly(N-(6-deoxy-D-galactose)acrylamide), a galactose-derived poly(N-((3,4,5,6-tetrahydroxytetrahydro-2H-pyran-2-yl)methyl)acrylamide), as a white solid.

### Example 7

### Collagen Hydrogels for Cartilage Growth and Repair

Soluble collagen is extracted and purified from animal sources and made non-antigenic. The pepsin-treated telopeptide poor collagen is lyophilized and stored at 20° C. for further use. A 2% collagen solution in 1 mM HCl (6 ml), is added to a glass tube, followed by N-(6-deoxy-D-galactose)acrylamide (2.6 g. from Example 1) with thorough mixing until a solution is formed. The w/w ratio of collagen to N-(6-deoxy-D-galactose)acrylamide is about 1:20. Propylene glycol (3 ml) is then added to the solution followed by 6% (w/v) aqueous ammonium persulfate (0.3 ml) and 12% (w/v) aqueous sodium metabisulfite (0.3 ml). All solutions are cooled to 3° C. before addition to the solution of collagen and N-(6-deoxy-D-galactose)acrylamide. The contents of the tube are thoroughly mixed and the polymerization reaction is allowed to proceed for 3 hours at room temperature.

### Example 8

### Anti-Cancer Drug-Containing Hydrogels

A. Methotrexate-containing hydrogels. A hydrogel containing methotrexate is prepared as follows. An aqueous solution of the methotrexate (25 mg methotrexate in 1 mL of pH 7.4 phosphate buffer, PBS) is added to a glass tube. N-(6-deoxy-D-galactose)acrylamide (2.6 g, from Example 1) in PBS (6 mL) is added to the tube, followed by N-((3,4,6-trihydroxy-5-methacrylamidotetrahydro-2H-pyran-2-yl)methyl)methacrylamide cross-linking agent (0.2 g, from Example 4). Propylene glycol (3 ml) is then added to the tube, followed by 6% (w/v) aqueous ammonium persulfate (0.3 ml) and 12% (w/v) aqueous sodium metabisulfite (0.3 ml). All solutions are cooled to 3° C. before addition to the tube. The contents of the tube are thoroughly mixed and the polymerization reaction is allowed to proceed for 3 hours at room temperature. The smooth, cylindrical and opaque hydrogel thus formed is washed exhaustively with PBS. A hydrogel containing methotrexate may also be prepared using collagen as a cross-linking agent, instead of N-((3,4,6-trihydroxy-5-methacrylamidotetrahydro-2H-pyran-2-yl)methyl)methacrylamide.

B. Cisplatin-containing hydrogels. A hydrogel containing cisplatin is prepared as follows. An aqueous solution of the cisplatin (20 mg cisplatin in 1 mL of water) is added to a glass tube. N-(6-deoxy-D-galactose)acrylamide (2.6 g, from Example 1) in water (6 mL) is added to the tube, followed by N-((3,4,6-trihydroxy-5-methacrylamidotetrahydro-2H-pyran-2-yl)methyl)methacrylamide cross-linking agent (0.2 g, from Example 5). Propylene glycol (3 ml) is then added to the tube, followed by 6% (w/v) aqueous ammonium persulfate (0.3 ml) and 12% (w/v) aqueous sodium metabisulfite (0.3 ml). All solutions are cooled to 3° C. before addition to the tube. The contents of the tube are thoroughly mixed and the polymerization reaction is allowed to proceed for 3 hours at room temperature. The smooth, cylindrical and opaque hydrogel thus formed is washed exhaustively with water. A hydrogel containing cisplatin may also be prepared using collagen as a cross-linking agent, instead of N-((3,4,6-trihydroxy-5-methacrylamidotetrahydro-2H-pyran-2-yl)methyl)methacrylamide.

### Example 9

### Hydrogel Scaffolds for Cell Growth

### A. Fibrous Mats Via Electrospinning

Fibrous mats of hydrogel materials may be obtained through simultaneous cross-linking and electrospinning. The electrospinning apparatus is equipped with a high-voltage statitron as in Yang. Y. et al. (“Electrospun Composite Mats of Poly[(D,Llactide)-co-glycolide] and Collagen with High Porosity as Potential Scaffolds for Skin Tissue Engineering” Macromolecular Materials and Engineering (2009), 294(9), 611-619.) N-(6-deoxy-D-glucose)acrylamide (prepared analogously to that described in Example 3 for N-(6-deoxy-D-glucose)methacrylamide, but using acryloyl chloride rather than methacroyl chloride) and N-((3,4,6-trihydroxy-5-methacrylamidotetrahydro-2H-pyran-2-yl)methyl)methacrylamide cross-linking agent from Example 4 are dissolved in water to prepare a 30 wt % solution, and the solution is added to a 2 mL glass syringe. The syringe is equipped with a metal capillary. The flow of the solution is controlled by a precision pump to maintain a steady flow of 0.5 mL/hour from the capillary outlet. The electrospun fibers are deposited on a rotating frame cylinder collector which includes metal struts. When using the frame including metal struts as the collector, the electrostatic forces drive the fibers to move towards the metal struts. Higher density fibers are deposited on the metal struts while lower density fibers are deposited between the struts. The rotating speed of the cylinder collector is controlled by a stepping motor. The deposition time can be optimized to obtain fibrous mats with thicknesses of 250-300 μm. The non-woven fibrous mat is vacuum-dried at room temperature for 3 days to completely remove any solvent residue prior to further characterization or use, e.g., in tissue engineering, including wound healing.

The above example may be modified by using collagen rather than N-((3,4,6-trihydroxy-5-methacrylamidotetrahydro-2H-pyran-2-yl)methyl)methacrylamide as the cross-linking agent. The hydrogel mat thus prepared can be used as hydrogels for cartilage repair.

### B. Scaffolds with Spherical Pores

Poly(methyl methacrylate) (PMMA) microspheres with diameter 90±10 μm may be manufactured as porogen templates essentially in accordance with Ivirico, J. L. et al., “Proliferation and differentiation of goat bone marrow stromal cells in 3D scaffolds with tunable hydrophilicity” J. Biomed. Mater. Res., Part B: Applied Biomaterials (2009), 91B(I), 277-286. The microspheres are introduced between two plates whose distance can be controlled by adjusting the step of a coupled screw. The plates are heated at 180° C. for 30 min to obtain the first template. The template shows the highest porosity attainable with typical compaction values of about 60-65% for random mono-sized spherical particles. To obtain scaffolds with controlled porosity, the thickness of the obtained disk is first measured; then the disk is replaced in the mold and compressed at 180° C. for 30 min. The degree of compression is quantified by measuring the thickness diminution.

A 40% solution of N-(6-deoxy-D-glucose)-acrylamide (98%) (that is prepared analogously to that described in Example 3 for N-(6-deoxy-D-glucose)methacrylamide, but using acryloyl chloride rather than methacroyl chloride) and N-((3,4,6-trihydroxy-5-methacrylamidotetrahydro-2H-pyran-2-yl)methyl)methacrylamide (Example 5) cross-linking agent (2%) in either water (6 mL) or PBS buffer (6 mL) is prepared. Ethylene glycol (3 ml) is added to the monomer solution followed by the addition of 6% (w/v) aqueous ammonium persulfate (0.5 mL) and 12% (w/v) aqueous sodium metabisulfite (0.5 mL). Cooling the monomer solution to 3° C. greatly increases working time. The monomer solution is then introduced in the empty space between the PMMA spheres of the template (after the template has been cooled to room temperature). Polymerization of the monomer solution by gentle warming of the template/solution at about 40° C. provides a hydrogel-porogen template composite. Alternatively, a UV photoinitiator can be used in place of the ammonium persulfate and sodium metabisulfite and the monomer solution may be polymerized using a UV source.

Porogen templated collagen-hydrogels are prepared using an analogous process, and can be used for cartilage growth and repair. High percentages (up to 50 wt % solutions) of N-((3,4,5,6-tetrahydroxytetrahydro-2H-pyran-2-yl)methyl)acrylamide or methacrylamide (i.e., such as those indicated in Examples 1-3) and collagen are used to create hydrogels with higher solids and cross-linking to make mechanically robust hydrogels. Soluble collagen is extracted and is purified from animal sources and made non-antigenic using known methods. The pepsin treated telopeptide poor collagen is lyophilized and stored at 20° C. for further use. Preparation of the hydrogel is carried out in a glass tube. A 20% collagen solution in 1 mM HCl (6 mL) is added, followed by N-(6-deoxy-D-glucose)acrylamide (3.2 g, prepared analogously to that described in Example 3 for N-(6-deoxy-D-glucose)methacrylamide, but using acryloyl chloride rather than methacroyl chloride) with thorough mixing. The collagen/N-(6-deoxy-D-glucose)acrylamide w/w ratio is about 1:20. Propylene glycol (3 ml) is added, followed by of DAROCUR 1173 photoinitiator (0.1 g). The contents are thoroughly mixed and then injected into the porogen template. The solution is then polymerized by exposure to UV light on all sides of the template for a period of about 5 minutes. A thermal initiator system (e.g., persulfate) can also be used in place of a photoinitiator.

After polymerization takes place, the PMMA porogen template is removed from the hydrogel by extraction with an organic solvent such as acetone or methylene chloride. The porous hydrogel sample is then extracted with ethanol to remove low molecular weight substances. Hydrogel samples are then dried in vacuum to constant weight before characterization. The cross-linked porous samples can be re-swelled with water or PBS buffer.

### C. Templating with Porous High Internal Phase Emulsion Polymers (polyHIPEs)

An aqueous phase of N-((3,4,5,6-tetrahydroxytetrahydro-2H-pyran-2-yl)methyl)acrylamide or methacrylamide (70 wt. %, such as those indicated in Examples 1-3), N-((3,4,6-trihydroxy-5-methacrylamidotetrahydro-2H-pyran-2-yl)methyl)methacrylamide cross-linking agent (0.9 wt. %, Example 4) is added to a 250 mL three-necked round bottomed flask, along with DAROCUR 1173 (0.1%) and sorbitan monooleate (0.05% surfactant). The mixture is stirred continually at 300 rpm using a D-shaped PTFE paddle connected to an overhead stirrer. Cyclohexane is then added over a period of two minutes using a peristaltic pump until the polyHIPE has formed. After addition of the cyclohexane, the polyHIPE is stirred for an additional one minute. The polyHIPE is then transferred to a glass centrifuge tube, which is irradiated with UV light for 5 minutes. Alternatively, a thermal initiator such as azobisisobutyronitrile can be used in lieu of the photoinitiator and the polyHIPE heated for about 24 hours at 60° C. The resulting monolith polymer is recovered from the glass tube. The monolith polymer is extracted with isopropanol in a Soxhlet apparatus for a period of 24 hours to remove low molecular weight impurities and is further dried under vacuum.

In a variation of this Example, collagen may be used as the cross-linker (in place of N-((3,4,6-trihydroxy-5-methacrylamidotetrahydro-2H-pyran-2-yl)methyl)methacrylamide) to prepare hydrogels for cartilage repair.

### EQUIVALENTS

The embodiments, illustratively described herein may suitably be practiced in the absence of any element or elements, limitation or limitations, not specifically disclosed herein. Thus, for example, the terms ‘comprising,’ ‘including,’ ‘containing,’ etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase ‘consisting essentially of’ will be understood to include those elements specifically recited and those additional elements that do not materially affect the basic and novel characteristics of the claimed technology. The phrase ‘consisting of’ excludes any element not specified.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent compositions, apparatuses, and methods within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as ‘up to,’ ‘at least,’ ‘greater than,’ ‘less than,’ and the like, include the number recited and refer to ranges which can be subsequently broken down into subranges as discussed above. Finally, as will be understood by one skilled in the art, a range includes each individual member.

While certain embodiments have been illustrated and described, it should be understood that changes and modifications can be made therein in accordance with ordinary skill in the art without departing from the technology in its broader aspects as defined in the following claims.

1. A hydrogel comprising a polyacrylamide wherein the polyacrylamide comprises one or more repeating units derived from the compound of Formula I: 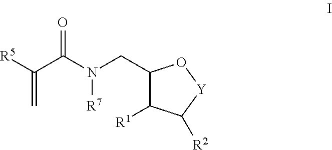
and stereoisomers thereof, wherein
Y is —C(H)(R³)— or —C(H)(R³)—C(H)(R⁴)—;
R¹, R², R³, and R⁴ are independently an —OH, a protected hydroxyl group, or a group of Formula II: 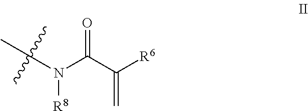
provided that not more than one of R¹, R², R³, and R⁴ is a group of Formula II; and
R⁵, R⁶, R⁷ and R⁸ are independently selected from hydrogen and a substituted or unsubstituted alkyl group.

2. The hydrogel of claim 1 wherein the protected hydroxyl group is selected from trimethylsilyl, t-butyldimethylsilyl, acetyl, benzyl, benzoyl, or methoxymethyl.

3. The hydrogel of claim 1 wherein R⁵ and R⁶ are independently selected from the group consisting of H and a methyl group.

4. (canceled)

5. (canceled)

6. The hydrogel of claim 1 wherein the one or more repeating units are derived from one or more compounds selected from
N-((3,4,5,6-tetrahydroxytetrahydro-2H-pyran-2-yl)methyl)acrylamide,
N-((3,4,5,6-tetrahydroxytetrahydro-2H-pyran-2-yl)methyl)methacrylamide,
N-((3,4,6-trihydroxy-5-methacrylamidotetrahydro-2H-pyran-2-yl)methyl)methacrylamide,
N-((5-acrylamido-3,4,6-trihydroxytetrahydro-2H-pyran-2-yl)methyl)methacrylamide,
N-(6-(acrylamidomethyl)-2,4,5-trihydroxytetrahydro-2H-pyran-3-yl)methacrylamide, or
N-((5-acrylamido-3,4,6-trihydroxytetrahydro-2H-pyran-2-yl)methyl)acrylamide.

7. (canceled)

8. (canceled)

9. (canceled)

10. (canceled)

11. (canceled)

12. (canceled)

13. (canceled)

14. (canceled)

15. (canceled)

16. (canceled)

17. A method of making a hydrogel comprising:
polymerizing one or more compounds of Formula I or stereoisomers thereof in an aqueous solution to form the hydrogel, wherein Formula I is 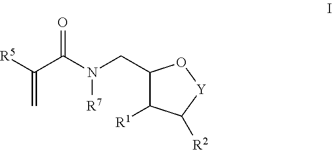
and
Y is —C(H)(R³)— or —C(H)(R³)—C(H)(R⁴)—;
R¹, R², R³, and R⁴ are independently an —OH, a protected hydroxyl group, or a group of Formula II: 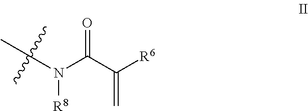
provided that not more than one of R¹, R², R³, and R⁴ is a group of Formula II; and
R⁵, R⁶, R⁷ and R⁸ are independently selected from the group consisting of hydrogen and a substituted or unsubstituted alkyl group.

18. The method of claim 17 wherein the protected hydroxyl group is selected from trimethylsilyl, t-butyldimethylsilyl, acetyl, benzyl, benzoyl, or methoxymethyl.

19. The method of claim 17 wherein the one or more compounds of Formula I are selected from compounds of Formula IA, IB or both IA and IB:

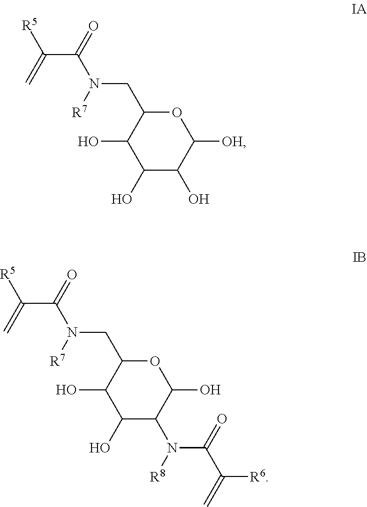

20. The method of claim 17 wherein the one or more repeating units are derived from one or more compounds selected from
N-((3,4,5,6-tetrahydroxytetrahydro-2H-pyran-2-yl)methyl)acrylamide,
N-((3,4,5,6-tetrahydroxytetrahydro-2H-pyran-2-yl)methyl)methacrylamide,
N-((3,4,6-trihydroxy-5-methacrylamidotetrahydro-2H-pyran-2-yl)methyl)methacrylamide,
N-((5-acrylamido-3,4,6-trihydroxytetrahydro-2H-pyran-2-yl)methyl)methacrylamide,
N-(6-(acrylamidomethyl)-2,4,5-trihydroxytetrahydro-2H-pyran-3-yl)methacrylamide, or
N-((5-acrylamido-3,4,6-trihydroxytetrahydro-2H-pyran-2-yl)methyl)acrylamide.

21. The method of claim 19 wherein the hydrogel comprises from about 90 wt % to about 99.9 wt % of the compound of Formula IA and from about 0.1 wt % to about 10 wt % of the compound of Formula IB, based on the total weight of the compounds of Formula IA and IB.

22. The method of claim 17 further comprising carrying out the polymerization in the presence of collagen.

23. (canceled)

24. (canceled)

25. (canceled)

26. The method of claim 17 wherein the polymerization is a high internal phase emulsion polymerization.

27. A non-woven fibrous mat of electrospun polyacrylamide wherein the polyacrylamide comprises one or more repeating units derived from the compound of Formula I: 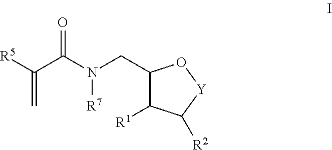
and stereoisomers thereof, wherein
Y is —C(H)(R³)— or —C(H)(R³)—C(H)(R⁴)—;
R¹, R², R³, and R⁴ are independently an —OH, a protected hydroxyl group, or a group of Formula II: 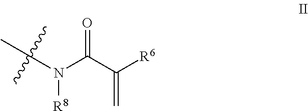
provided that not more than one of R¹, R², R³, and R⁴ is a group of Formula II; and
R⁵, R⁶, R⁷ and R⁸ are independently selected from the group consisting of hydrogen and a substituted or unsubstituted alkyl group.

28. The non-woven fibrous mat of claim 27 comprising cells entrained within the mat.

29. The non-woven fibrous mat of claim 28 wherein the cells are selected from the group consisting of bone marrow, neuroblastoma, adenocarcinoma, carcinoma, colorectal carcinoma, glioblastoma, fibroblast, and myeloblast cells.

30. A method of wound healing or tissue regeneration comprising implanting the non-woven fibrous mat of claim 27 into a patient in need thereof.
